SEQUENCE LISTING (41 sequences)

SEQ ID NO: 1 - RNA, 21 nt, Epstein Barr Virus
aaccugaucagccccggaguu

SEQ ID NO: 2 - RNA, 23 nt, Epstein Barr Virus
uaucuuuugcggcagaaauugaa

SEQ ID NO: 3 - RNA, 23 nt, Epstein Barr Virus
uaacgggaaguguguaagcacac

SEQ ID NO: 4 - RNA, 21 nt, Epstein Barr Virus
ucuuaguggaagugacgugcu

SEQ ID NO: 5 - RNA, 21 nt, Epstein Barr Virus
ucuuaguggaagugacgugcu

SEQ ID NO: 6 - RNA, 22 nt, Kaposi Sarcoma Associated Virus
uaguguuguccccccgaguggc

SEQ ID NO: 7 - RNA, 22 nt, Kaposi Sarcoma Associated Virus
ugguguuguccccccgaguggc

SEQ ID NO: 8 - RNA, 22 nt, Kaposi Sarcoma Associated Virus
cuggguauacgcagcugcguaa

SEQ ID NO: 9 - RNA, 22 nt, Kaposi Sarcoma Associated Virus
uaggcgcgacugagagagcacg

SEQ ID NO: 10 - RNA, 21 nt, Kaposi Sarcoma Associated Virus
ugaucccauguugcuggcgcu

SEQ ID NO: 11 - RNA, 22 nt, Kaposi Sarcoma Associated Virus
uuaaugcuuagccuguguccga

SEQ ID NO: 12 - RNA, 22 nt, Kaposi Sarcoma Associated Virus
ccagcagcaccuaauccaucgg

SEQ ID NO: 13 - RNA, 22 nt, Kaposi Sarcoma Associated Virus
uaggaugccuggaacuugccgg

SEQ ID NO: 14 - RNA, 22 nt, Kaposi Sarcoma Associated Virus
agcuaaaccgcaguacucuagg

SEQ ID NO: 15 - RNA, 22 nt, Kaposi Sarcoma Associated Virus
ucacauucugaggacggcagcg

SEQ ID NO: 16 - RNA, 23 nt, Kaposi Sarcoma Associated Virus
auuacaggaaacuggguguaagc

SEQ ID NO: 17 - RNA, 20 nt, Human cytomegalovirus
uaacuagccuucccgugaga

SEQ ID NO: 18 - RNA, 22 nt, Human cytomegalovirus
ucguugaagacaccuggaaaga

SEQ ID NO: 19 - RNA, 22 nt, Human cytomegalovirus
aagugacggugagauccaggcu

SEQ ID NO: 20 - RNA, 21 nt, Human cytomegalovirus
ucguccuccccuucuucaccg

SEQ ID NO: 21 - RNA, 21 nt, Human cytomegalovirus
ugacaagccugacgagagcgu

SEQ ID NO: 22 - RNA, 22 nt, Human cytomegalovirus
uuaugauaggugugacgauguc

SEQ ID NO: 23 - RNA, 21 nt, Human cytomegalovirus
aaccgcucaguggcucggacc

SEQ ID NO: 24 - RNA, 22 nt, Human cytomegalovirus
agcggucuguucagguggauga

SEQ ID NO: 25 - RNA, 22 nt, Human cytomegalovirus
gauugugcccggaccgugggcg

SEQ ID NO: 26 - RNA, 22 nt, Human immunodeficiency virus type 1
ugggucucucugguuagaccag

SEQ ID NO: 27 - RNA, 22 nt, Human immunodeficiency virus type 1
cucucuggcuaacuagggaacc

SEQ ID NO: 28 - RNA, 22 nt, Human immunodeficiency virus type 1
cccuauagugcagaaccuccag

SEQ ID NO: 29 - RNA, 22 nt, Human immunodeficiency virus type 1
ccugaacuuuaaaugcauggga

SEQ ID NO: 30 - RNA, 22 nt, Human immunodeficiency virus type 1
uuuagggaagaucuggccuucc

SEQ ID NO: 31 - RNA, 22 nt, Human immunodeficiency virus type 1
gggaaggccagggaauuuucuu

SEQ ID NO: 32 - RNA, 22 nt, Human immunodeficiency virus type 1
ccugagagagaaguguuagagu

SEQ ID NO: 33 - RNA, 22 nt, Human immunodeficiency virus type 1
cuagcauuucaucacguggccc

SEQ ID NO: 34 - RNA, 22 nt, Human immunodeficiency virus type 1
gggaacccacugcuuaagccuc

SEQ ID NO: 35 - RNA, 22 nt, Human immunodeficiency virus type 1
uucaaguagugugugcccgucu

SEQ ID NO: 36 - RNA, 69 nt, Human immunodeficiency virus type 1
guacugggucucucugguuagaccagaucugagccuggagcucucuggcuaacuagggaacccacugcu

SEQ ID NO: 37 - RNA, 76 nt, Human immunodeficiency virus type 1
uuacccuauagugcagaaccuccaggggcaaaugguuuuucaggccauaucaccugaacuuuaaaugcauggguaa

SEQ ID NO: 38 - RNA, 76 nt, Human immunodeficiency virus type 1
ggcuaauuuuuuagggaagaucuggccuuccccccagggaaggccagggaauuuucuucagagcagaccagagcca

SEQ ID NO: 39 - RNA, 71 nt, Human immunodeficiency virus type 1
uggaugacccugagagagaaguguuagaguggagguuugacagccgccuagcauuucaucacguggcccga

SEQ ID NO: 40 - RNA, 75 nt, Human immunodeficiency virus type 1
uaacuagggaacccacugcuuaagccucaauaaagcuugccuugagugcuucaaguagugugugcccgucuguug

SEQ ID NO: 41 - RNA, 22 nt, Human immunodeficiency virus type 1
ccugaacuuuaaaugcaugggu

(19) United States
(12) Patent Application Publication
(10) Pub. No.: US 2006/0293267 A1
(43) Pub. Date: Dec. 28, 2006

(54) Dual functional oligonucleotides for use as anti-viral agents

(75) Inventors: Phillip D. Zamore, Northboro, MA (US); Jennifer Broderick, Cambridge, MA (US)

Correspondence Address: LAHIVE & COCKFIELD, LLP, ONE POST OFFICE SQUARE, BOSTON, MA, 02109-2127, US

(73) Assignee: UNIVERSITY OF MASSACHUSETTS, Boston, MA (US)

(21) Appl. No.: 11/405,028

(22) Filed: Apr. 13, 2006

Related U.S. Application Data
(60) Provisional application No. 60/671,356, filed on Apr. 13, 2005.

(51) Int. Cl. A61K 48/00; C12Q 1/70; C07H 21/02

(52) U.S. Cl. 514/44; 536/23.1; 435/5

(57) ABSTRACT

The present invention is based, in part, on the discovery that endogenous mRNAs, such as viral miRNAs, can be recruited for translational repression of target mRNAs, such as viral target mRNAs. The RNA-silencing agents and the methods described herein, thereby provide a means of treating viral infections, of treating diseases or disorders caused by viral infections, or for preventing viral propagation. The RNA-silencing agents of the present invention have an mRNA targeting moiety, a linking moiety, and a viral miRNA recruiting moiety.

### RELATED APPLICATIONS

This application claims the benefit of U.S. Ser. No. 60/671,356, entitiled “Dual Functional Oligonucleotides For Use As Anti-Viral Agents”, filed on Apr. 13, 2005. The entire contents of this application are hereby incorporated herein by reference.

The contents of any patents, patent applications, and references cited throughout this specification are hereby incorporated by reference in their entireties.

### BACKGROUND OF THE INVENTION

RNA silencing refers to a group of sequence-specific regulatory mechanisms (e.g. RNA interference (RNAi), transcriptional gene silencing (TGS), post-transriptional gene silencing (PTGS), quelling, co-suppression, and translational repression) mediated by RNA molecules which result in repression or “silencing” of a corresponding protein-coding gene. RNA silencing has been observed in many types of organisms, including plants, animals, and fungi.

Two types of small (˜19-23 nt), noncoding RNAs trigger RNA silencing in eukaryotes: small interfering RNAs (siRNAs) and microRNAs (miRNAs, also known as small temporal RNAs (stRNAs)). Both siRNAs and miRNAs are produced by the cleavage of double-stranded RNA (dsRNA) precursors by Dicer, a nuclease of the RNase III family of dsRNA-specific endonucleases (Bernstein et al., 2001; Billy et al., 2001; Grishok et al., 2001; Hutvágner et al., 2001; Ketting et al., 2001; Knight and Bass, 2001; Paddison et al., 2002; Park et al., 2002; Provost et al., 2002; Reinhart et al., 2002; Zhang et al., 2002; Doi et al., 2003; Myers et al., 2003).

siRNAs result when transposons, viruses or endogenous genes express long dsRNA or when dsRNA is introduced experimentally into plant or animal cells to associate with and guide a protein complex called RNA-induced silencing complex (RISC) to direct the sequence-specific destruction of a complementary target mRNA by endonucleolytic cleavage, a process known as RNA interference (RNAi) (Fire et al., 1998; Hamilton and Baulcombe, 1999; Zamore et al., 2000; Elbashir et al., 2001a; Hammond et al., 2001; Sijen et al., 2001; Catalanotto et al., 2002). In contrast, miRNAs are the products of endogenous, non-coding genes whose transcripts form long, largely single-stranded RNA transcripts termed pri-miRNAs. Pri-miRNAs are sequentially processed, first in the nucleus by Drosha to form a ˜65 nt stem-loop RNA precursor termed a pre-miRNA, then in the cytoplasm by Dicer to form mature mRNAs of 21-23 nucleotides (Lagos-Quintana et al., 2001; Lau et al., 2001; Lee and Ambros, 2001; Lagos-Quintana et al., 2002; Mourelatos et al., 2002; Reinhart et al., 2002; Ambros et al., 2003; Brennecke et al., 2003; Lagos-Quintana et al., 2003; Lim et al., 2003a; Lim et al., 2003b). Although, miRNAs exist transiently in the cell as double-stranded molecules, one strand (usually the antisense strand) is incorporated into RISC while the other strand (usually the sense strand) is rapidly degraded.

Recent evidence has suggested that mRNAs mediate RNA silencing by distinct but interchangeable mechanisms which are determined, among other factors, by the degree of complementarity between the small RNA and its target mRNA (Schwarz and Zamore, 2002; Hutvágner and Zamore, 2002; Zeng et al., 2003; Doench et al., 2003). miRNAs with a high degree of complementarity to a corresponding target mRNA have been shown to direct its cleavage by the RNAi mechanism (Zamore et al., 2000; Elbashir et al., 2001a; Rhoades et al., 2002; Reinhart et al., 2002; Llave et al., 2002a; Llave et al., 2002b; Xie et al., 2003; Kasschau et al., 2003; Tang et al., 2003; Chen, 2003). miRNAs with a lower degree of complementarity mediate gene silencing by recruiting the RISC complex to the target mRNA, thereby blocking its translation but leaving the mRNA intact (Mourelatos et al., 2002; Hutvágner and Zamore, 2002; Caudy et al., 2002; Martinez et al., 2002; Abrahante et al., 2003; Brennecke et al., 2003; Lin et al., 2003; Xu et al., 2003).

Since their discovery in plant and animals, miRNAs have been ascribed diverse physiological roles, including the regulation of developmental-timing, cell proliferation, cell death, and fat metabolism (see, for example, Carrington and Ambros, 2003; Baehrecke, 2003). Recently, viruses have also been shown to express miRNAs (Pfeffer et al, 2004). However, the precise role played by viral miRNAs in infectious disease has yet to be elucidated. Moreover, the potential of viral miRNA to affect and control host-pathogen interactions (e.g., those associated with infectious diseases or disorders) is yet to be harnessed in an effective and efficient manner.

### SUMMARY OF THE INVENTION

The present invention is based, in part, on the discovery that the mRNA expressed by a virus can be recruited by an RNA-silencing agent to silence the expression of a target mRNA in a cell infected with said virus. The RNA-silencing agents of the present invention serve to bring viral miRNAs within the vicinity of the target mRNA so as to promote RNA silencing of the target mRNA. Since the RNA-silencing agents can only induce RNA silencing in a cell where both the viral miRNA and target mRNA are co-expressed, and further, since viral miRNAs are only expressed in cells infected with the virus encoding them, said agents may be employed as inter alia highly effective anti-viral agents.

In one aspect, the invention provides an RNA-silencing agent having the formula T-L-Vμ, where T is an mRNA targeting moiety, L is a linking moiety, and Vμis a viral miRNA recruiting moiety. In another aspect, the invention provides an RNA silencing agent suitable for use in gene silencing of a target mRNA, having an mRNA targeting portion complementary to the target mRNA; a viral miRNA recruiting portion complementary to a viral miRNA; and a linking portion that links the mRNA targeting portion and the mRNA recruiting portion.

In one embodiment, the RNA-silencing agent includes an mRNA targeting moiety or portion of about 9 to about 24 nucleotides in length (for example, 15 nucleotides in length). In another embodiment, the RNA-silencing agent includes a viral miRNA recruiting moiety or portion that is about 13 to about 21 nucleotides in length (for example, about 13 or about 15 nucleotides in length).

In one embodiment, the target mRNA is a host mRNA that is expressed by a host cell infected with a virus. In certain embodiments, said host mRNA is necessary for the productive infection of the host by the virus. In other embodiments, the host mRNA is encoded by a host gene that is necessary for the survival of the host cell.

In another embodiment, the target mRNA is a viral mRNA that is expressed by a virus upon infection of the host cell. In certain embodiment, said viral mRNA is necessary for the productive infection of the host by the virus.

In another embodiment, the mRNA targeting moiety or portion targets an mRNA encoding a protein involved in infectious disease (e.g., AIDS) or disorder. In yet another embodiment, the mRNA targeting moiety or portion targets an mRNA encoding a viral receptor (e.g., CCR5).

In one embodiment, the linking moiety or portion is a phosphodiester bond. In one embodiment, the linking moiety or portion includes at least one modified nucleotide which increases the in vivo stability of the agent. For example, the linking moiety or portion has at least one 2′-O-methyl nucleotide and/or at least one phosphorothioate nucleotide. In another embodiment, the linking moiety or portion has at least one locked nucleotide (e.g., C2′-O,C4′-ethylene-bridged nucleotide). In other embodiments, the linking moiety or portion has at least one sugar-modified nucleotide and/or at least one base-modified nucleotide.

In another embodiment, the viral miRNA recruiting moiety or portion recruits a viral miRNA capable of inducing RNA silencing via a RNA-induced silencing complex (RISC). In another embodiment, the miRNA recruiting moiety or portion recruits an miRNA selected from the group consisting of:
a) a nucleotide sequence as shown in Table 1;
b) a nucleotide sequence which is the complement of (a);
c) a nucleotide sequence which has an identity of at least 80%, preferably of at least 90%, and more preferably of at least 99%, to a sequence of (a) or (b); and
d) a nucleotide sequence which hybridizes under stringent conditions to a sequence of (a), (b), and/or (c).

In yet another embodiment, the miRNA recruiting moiety or portion recruits an HIV miRNA, a herepesvirus miRNA, or a adenoviral miRNA.

In yet another embodiment, the invention provides a composition including an RNA-silencing agent and a pharmaceutically acceptable carrier.

In another aspect, the invention provides DNA constructs encoding said RNA-silencing agents. In one embodiment, the construct is a plasmid.

In another aspect, the invention provides a method of inducing RNA silencing of a gene (e.g., a gene encoding a protein, for example, a protein associated with a viral disease or a disorder) in a cell containing a viral miRNA, including contacting a cell with an RNA-silencing agent, under conditions such that the agent induces RNA silencing within the cell (e.g., in an organism).

In yet another aspect, the invention provides a method for treating a subject having or at risk for an infectious disease or disorder characterized or caused by the overexpression or overactivity of a cellular protein, including administering to the subject an effective amount of an RNA-silencing agent, wherein the mRNA targeting moiety targets an mRNA encoding said protein.

In yet another aspect, the invention provides a method for treating a subject having or at risk for an infectious disease (e.g., AIDS) or disorder characterized or caused by a virus, including administering to the subject an effective amount of an RNA-silencing agent, wherein the viral miRNA recruiting moiety targets a viral miRNA expressed by said virus.

In another aspect, the invention provides for the use of an RNA-silencing agent in the manufacture of a medicament for the prevention or treatment of infectious disease.

### BRIEF DESCRIPTION OF THE DRAWINGS

FIG. 1 depicts the recruitment of a viral miRNA using the RNA-silencing agents of the present invention. FIG. 1A depicts an RNA-silencing agent and a viral miRNA associated with the protein complex, RISC. FIG. 1B depicts the RNA-silencing agent associating with the target mRNA, luciferase, and the viral miRNA to mediate translational repression of the target mRNA.

FIG. 2 depicts mRNAs associated with HIV. FIG. 2A identifies the location of the coding sequences on the HIV genome. FIG. 2B depicts the predicted precursor structures (SEQ ID NOS: 36-40, respectively, in order of appearance), mature viral miRNA sequences (SEQ ID NOS: 26-28, 41 and 30-35, respectively, in order of appearance) and their localization on the HIV genome.

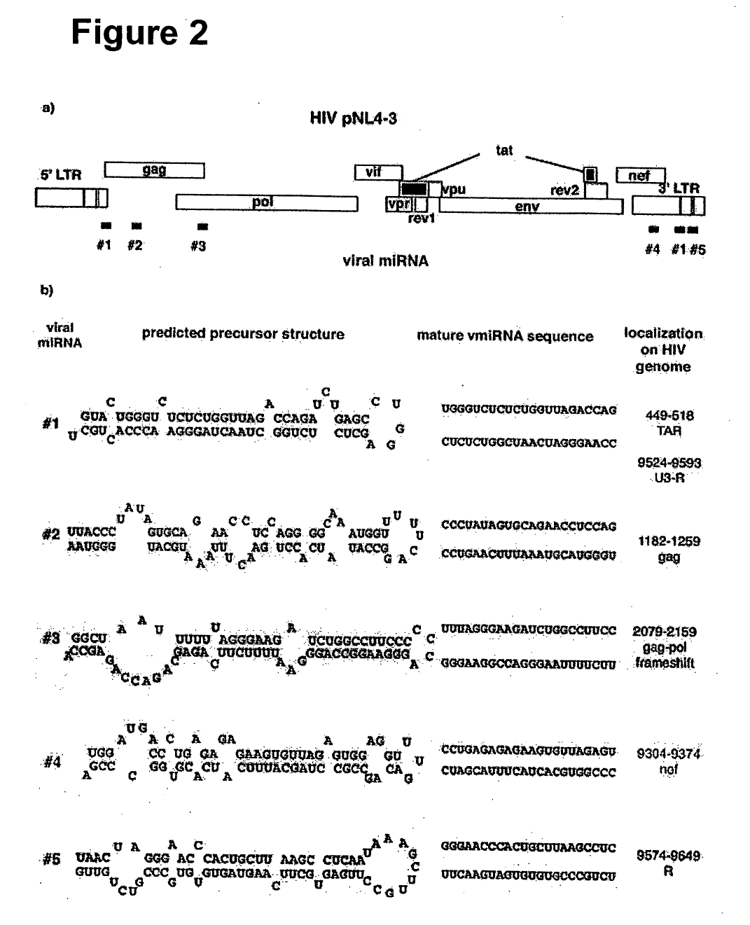

### DETAILED DESCRIPTION OF THE INVENTION

The present invention is based, in part, on the discovery that viral miRNAs can be recruited for gene silencing of target mRNAs. Accordingly, RNA-silencing agents having an mRNA targeting moiety or portion, a linking moiety or portion, and an miRNA recruiting moiety or portion, are designed to promote RNA silencing of a target mRNA. The RNA-silencing agents and the methods described herein, thereby provide a means to treat or prevent infection by, transmission, and/or propagation of a virus expressing the viral miRNA. In addition, the RNA-silencing agents and the methods of the invention may be employed in the prevention or treatment of infectious diseases or disorders characterized by viruses which express said viral miRNAs. For example, the RNA-silencing agents and methods described herein may be used as anti-viral agents which are capable of preventing viral transmission or infection in a cell infected with a virus such as Human Immunodeficiency Virus (HIV) or Epstein Barr virus.

The methods of the present invention offer several advantages over existing gene silencing techniques to inhibit a productive viral infection. First, the methods described herein allow a molecule expressed solely in virally infected tissues, a viral miRNA, to mediate RNA silencing solely in said infected tissues. Secondly, the viral miRNA can be recruited to mediate RNA silencing of an mRNA to which the viral miRNA is non-complementary and whose silencing is adverse to viral infection, replication, and/or propagation. Thirdly, by recruiting said viral miRNA, the methods of the invention prevent the viral miRNA from performing a function which produces an environment conducive to viral infection, e.g. RNA silencing of a host gene involved in an antiviral response. Fourthly, the RNA-silencing agents, and their respective moieties, can be designed to conform to specific host and/or viral mRNA sites and specific viral miRNAs. The designs can be cell and gene product specific. Accordingly, RNA-silencing agents designed in accordance with the present invention can serve to selectively target different viruses, as well as different phases of a viral life cycle.

Definitions

So that the invention may be more readily understood, certain terms are first defined.

As used herein, the term “RNA-silencing agent” refers to a molecule having the formula T-L-Vμ, wherein T is an mRNA targeting moiety, L is a linking moiety, and Vμ is a viral miRNA recruiting moiety.

As used herein, the terms “mRNA targeting moiety”, “targeting moiety”, “mRNA targeting portion” or “targeting portion” refer to a domain, portion or region of the RNA-silencing agent having sufficient size and sufficient complementarity to a portion or region of an mRNA chosen or targeted for silencing (i.e., the moiety has a sequence sufficient to capture the target mRNA).

As used herein, the terms “viral miRNA recruiting moiety”, “viral recruiting moiety”, “viral miRNA recruiting portion” or “viral recruiting portion” refer to a domain, portion or region of the RNA-silencing agent having a sufficient size and sufficient complementarity to a viral miRNA (e.g., an miRNA encoded in a viral genome), or portion or region of said miRNA (i.e., the moiety has a sequence sufficient to recruit miRNA).

As used herein, the term “microRNA” (“miRNA”), also referred to in the art as a “small temporal RNA” (“stRNA”), refers to a small (10-50 nucleotide, e.g. a 21-23 nucleotide) RNA which is capable of directing or mediating RNA silencing. A “viral miRNA” refers to a microRNA that is encoded in a viral genome.

As used herein, the term “linking moiety” or “linking portion” refers to a domain, portion or region of the RNA-silencing agent which covalently joins or links the mRNA targeting moiety and the viral miRNA recruiting moiety.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide while retaining the ability of the nucleotide analog to perform its intended function.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain. chemical properties of the nucleotide yet retain the ability of the nucleotide analog to perform its intended function. Examples of preferred modified nucleotides include, but are not limited to, 2-amino-guanosine, 2-amino-adenosine, 2,6-diamino-guanosine and 2,6-diamino-adenosine. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2): 117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. The oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA silencing. In an exemplary embodiment, oligonucleotides comprise Locked Nucleic Acids (LNAs) or Peptide Nucleic Acids (PNAs).

As used herein, the term “RNA interference” (“RNAi”) refers to a type of RNA silencing which results in the selective intracellular degradation of a target mRNA. As used herein, the term “translational repression” refers to a type of RNA silencing which results in the selective inhibition of mRNA translation without selective intracellular degradation of a target mRNA. Both RNAi and translational repression are mediated by RISC. Both RNAi and translational repression occur naturally or can be initiated by the hand of man, for example, to silence the expression of target genes.

As used herein, the terms “sufficient complementarity” or “sufficient degree of complementarity” mean that the mRNA targeting moiety or the viral miRNA recruiting moiety has a sequence sufficient to bind the desired target mRNA or viral miRNA, respectively, and to trigger the RNA silencing of the target mRNA.

The term “mismatch” refers to a base pair consisting of noncomplementary bases, for example, not normal complementary G:C, A:T or A:U base pairs.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

A “target mRNA” refers to an mRNA (e.g., a viral mRNA or host cell mRNA) to which the mRNA targeting moiety is complementary and for which RNA silencing is desirable. A “target gene” is a gene encoding said target mRNA.

As used herein the phrase “early stages of a viral life cycle” means the stages of viral replication that occur up to and including replication of the viral genome and the phrase “late stages of a viral life cycle” means the stages of replication that occur following replication of the viral genome. Events exemplifying early stages of viral replication include, but are not limited to, attachment or adsorption of the virus to the cell, penetration of the host cell membrane by the virus, uncoating the viral capsid from the viral genome, Events exemplifying late stages of replication include, but are not limited to, integration of the viral DNA into the host cell's chromosome, production of viral RNAs, translation of viral proteins, and release of virions.

“Treatment”, or “treating” as used herein, is defined as the application or administration of a therapeutic agent (e.g., a RNA silencing agent or a vector or transgene encoding same) to a patient, or application or administration of a therapeutic agent to an isolated tissue or cell line from a patient, who has a virus with the purpose to cure, heal, alleviate, relieve, alter, remedy, ameliorate, improve or affect the virus, or symptoms of the virus. The term “treatment” or “treating” is also used herein in the context of administering agents prophylactically, e.g., to inoculate against a virus. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “patient” includes human and other mammalian subjects that receive either prophylactic or therapeutic treatment.

RNA-Silencing Agents

The present invention relates to RNA-silencing agents. The RNA-silencing agents of the invention are designed such that they recruit viral miRNAs to a target mRNA so as to induce RNA silencing. In preferred embodiments, the RNA-silencing agents have the formula T-L-Vμ, wherein T is an mRNA targeting moiety, L is a linking moiety, and Vμis a viral miRNA recruiting moiety. Any one or more moiety may be double stranded. Preferably, however, each moiety is single stranded.

Moieties within the RNA-silencing agents can be arranged or linked (in the 5′ to 3′ direction) as depicted in the formula T-L-Vμ (i.e., the 3′ end of the targeting moiety linked to the 5′ end of the linking moiety and the 3′ end of the linking moiety linked to the 5′ end of the viral miRNA recruiting moiety). Alternatively, the moeities can be arranged or linked in the RNA-silencing agent as follows: Vμ-T-L (i.e., the 3′ end of the viral miRNA recruiting moiety linked to the 5′ end of the linking moiety and the 3′ end of the linking moiety linked to the 5′ end of the targeting moiety).

a) Viral mRNA Targeting Moiety (Vμ)

The viral miRNA recruiting moiety, as described above, is capable of associating with a viral miRNA. According to the invention, the viral miRNA may be any viral miRNA expressed by a virus, including without limitation, miRNAs expressed by insect viruses, mammalian viruses, and plant viruses. Preferably, said viral miRNAs are capable of associating with the RISC complex.

In one embodiment, the viral miRNA is expressed by a double-stranded DNA virus. In another embodiment, the viral miRNA is expressed by a single-stranded DNA virus. In another embodiment, the viral miRNA is expressed by a double-stranded RNA virus. In another embodiment, the viral miRNA is expressed by a single-stranded (plus-strand) RNA virus. In another embodiment, the viral miRNA is expressed by a single-stranded (minus-strand) RNA virus. In another embodiment, the viral miRNA is expressed by a retrovirus.

In exemplary embodiments, the viral miRNA is expressed by a virus capable of infecting human cells. Such viruses include:
a) herpesviruses such as the simplexviruses (e.g. human herpesvirus-1 (HHV-1), human herpesvirus-2 (HHV-2)), the varicelloviruses (e.g. human herpesvirus-3 (HHV-3, also known as varicella zoster virus)), the lymphocryptoviruses (e.g. human herpesvirus-4 (HHV-4, also known as Epstein Barr virus (EBV))), the cytomegaloviruses (e.g. human herpesvirus-5 (HHV-5), also known as human cytomegalovirus (HCMV)), the roseoloviruses (e.g. human herpesvirus 6 (HHV-6), human herpesvirus 7 (HHV-7)), the rhadinovirues (e.g. human herpesvirus 8 (HHV-8, also known as Kaposi's Sarcoma associated herpesvirus (KSHV));
b) poxviruses such as orthopoxviruses (e.g. cowpoxvirus, monkeypoxvirus, vaccinia virus, variola virus), parapoxviruses (e.g. bovine popular stomatitis virus, orf virus, pseudocowpox virus), molluscipoxviruses (e.g. molluscum contagiosum virus), yatapoxviruses (e.g., tanapox virus, yaba monkey tumor virus);
c) adenoviruses (e.g. Human adenovirus A (HAdV-A), Human adenovirus B (HAdV-B), Human adenovirus C (HAdV-C), Human adenovirus D (HAdV-D), Human adenovirus E (HAdV-E), Human adenovirus F (HAdV-F));
d) papillomaviruses (e.g. human papillomavirus (HPV);
e) parvoviruses (e.g. B19 virus);
f) hepadnoviruses (e.g., Hepatitis B virus (HBV));
g) retroviruses such as deltaretroviruses (e.g. primate T-lymphotrophic virus 1 (HTLV-1) and primate T-lymphotrophic virus 2 (HTLV-2)) and lentiviruses (e.g. Human Immunodeficiency Virus 1 (HIV-1) and Human Immunodeficiency Virus 2 (HIV-2);
h) reoviruses such the orthoreoviruses (e.g. mammalian orthoreovirus (MRV)), the orbviruses (e.g. African horse sickness virus (AHSV), Changuinola virus (CORV), Orungo virus (ORUV), and the rotaviruses (e.g. rotavirus A (RV-A) and rotavirus B (RV-B));
i) filoviruses such as the “Marburg-like viruses” (e.g. MARV), the “Ebola-like viruses” (e.g. CIEBOV, REBOV, SEBOV, ZEBOV),
j) paramyxoviruses such as respiroviruses (e.g. human parainfluenza virus 1 (HPIV-1), human parainfluenza virus 3 (HPIV-3), rubulaviruses (e.g. human parainfluenza virus 2 (HPIV-2), human parainfluenza virus 4 (HPIV-4)), mumps virus (MuV)), and morbilliviruses (e.g. measles virus);
k) pneumoviruses (e.g. human respiratory syncitial virus (HSCV);
l) rhabdoviruses such as the vesiculoviruses (e.g. vesicular stomatitis virus), the lyssaviruses (e.g., rabies virus);
m) orthomyxoviruses (e.g. Influenza A virus, Influenza B virus, Influenza C virus);
n) bunyaviruses (e.g. California encephalitis virus (CEV));
o) hantaviruses (e.g. Black Creek Canal virus (BCCV), New York virus (NYV), Sin Nombre virus (SNV));
p) picornaviruses including the enteroviruses (e.g. human enterovirus A (HEV-A), human enterovirus B (HEV-B), human enterovirus C (HEV-C), human enterovirus D (HEV-D), poliovirus (PV)), the rhinoviruses (e.g. human rhinovirus A (HRV-A), human rhinovirus B (HRV-B)), the hepatoviruses (e.g. Hepatitis A virus (HAV));
q) caliciviruses including the “Norwalk-like viruses” (e.g. Norwalk Virus (NV), and the “Sapporo-like viruses” (e.g. Sapporo virus (SV));
r) togaviruses including alphaviruses (e.g. Western equine encephalitis virus (WEEV) and Eastern equine encephalitis virus (EEEV)) and rubiviruses (e.g. Rubella virus);
s) flaviviruses (e.g. Dengue virus (DENV), Japanese encephalitis (JEV), St. Louis encephalitis virus (SLEV), West Nile virus (WNV), Yellow fever virus (YFV);
t) arenaviruses (e.g. lassa virus);
u) coronaviruses (e.g. the severe acute respiratory syndrome (SARS)-associated virus); and
v) hepaciviruses (e.g. Hepatitis C virus (HCV)).

In various embodiments, the viral miRNA may be any art-recognized viral miRNA. Several viruses of the herpesvirus superfamily (e.g. Epstein Barr Virus, Kaposi's Sarcoma virus, and Human Cytomegalovirus) have recently been cloned (Pfeffer et al, Science. (2004), 304:734-736; Pfeffer et al., Nature Methods, (2005), 2(4): 269-276; Cai et al., Proc. Natl. Acad. Sci., (2005), 102: 5570-5575). In addition, several miRNA precursors have also been predicted to reside in the HIV-1 genome (Bennasser et al. (2004) Retrovirology. 1(1):43)). Table 1 lists some of these viral miRNAs.

TABLE 1
Viral miRNAs

| | | | | | | SEQ ID |
|---|---|---|---|---|---|---|
| ID | Virus | Gene | miRNA sequence (5′-3′) | Mature | Precursor | NO: |
| ebv-miR- | Epstein | miR- | aaccugaucagccccggaguu | 22 | 66 | 1 |
| BHRF1-1 | Barr Virus | BHRF1-1 | | | | |
| ebv-miR- | Epstein | miR- | uaucuuuugcggcagaaauugaa | 22/23 | 65 | 2 |
| BHRF1-2 | Barr Virus | BHRF1-2 | | | | |
| ebv-miR- | Epstein | miR- | uaacgggaaguguguaagcacac | 23 | 65 | 3 |
| BHRF1-3 | Barr Virus | BHRF1-3 | | | | |
| ebv-miR- | Epstein | miR- | ucuuaguggaagugacgugcu | 21 | 70 | 4 |
| BART1 | Barr Virus | BART1 | | | | |
| ebv-miR- | Epstein | miR- | ucuuaguggaagugacgugcu | 21 | 62 | 5 |
| BART2 | Barr Virus | BART2 | | | | |
| KSHV- | Kaposi | miR-K12- | uuaaugcuuagccuguguccga | 22 | 71 | 11 |
| miR K12- | Sarcoma | 11 | | | | |
| 11 | Associated | | | | | |
| | Virus | | | | | |
| KSHV-miR- | Kaposi | miR-K12- | uaguguuguccccccgaguggc | 22 | 70 | 6 |
| K12-10a | Sarcoma | 10a | | | | |
| | Associated | | | | | |
| | Virus | | | | | |
| KSHV-miR- | Kaposi | miR-K12- | ugguguuguccccccgaguggc | 22 | 70 | 7 |
| K12-10b | Sarcoma | 10b | | | | |
| | Associated | | | | | |
| | Virus | | | | | |
| KSHV-miR- | Kaposi | miR-K12-9 | cuggguauacgcagcugcguaa | 22 | 66 | 8 |
| K12-9 | Sarcoma | | | | | |
| | Associated | | | | | |
| | Virus | | | | | |
| KSHV- | Kaposi | miR-K12-8 | uaggcgcgacugagagagcacg | 22 | 70 | 9 |
| miR K12-8 | Sarcoma | | | | | |
| | Associated | | | | | |
| | Virus | | | | | |
| KSHV- | Kaposi | miR-K12-7 | ugaucccauguugcuggcgcu | 21 | 72 | 10 |
| miR K12-7 | Sarcoma | | | | | |
| | Associated | | | | | |
| | Virus | | | | | |
| KSHV- | Kaposi | miR-K12-6 | ccagcagcaccuaauccaucgg | 22 | 62 | 12 |
| miR K12-6 | Sarcoma | | | | | |
| | Associated | | | | | |
| | Virus | | | | | |
| KSHV- | Kaposi | miR-K12-5 | uaggaugccuggaacuugccgg | 22 | 70 | 13 |
| miR K12-5 | Sarcoma | | | | | |
| | Associated | | | | | |
| | Virus | | | | | |
| KSHV- | Kaposi | miR-K12-4 | agcuaaaccgcaguacucuagg | 22 | 70 | 14 |
| miR K12-4 | Sarcoma | | | | | |
| | Associated | | | | | |
| | Virus | | | | | |
| KSHV- | Kaposi | miR-K12-3 | ucacauucugaggacggcagcg | 22 | 70 | 15 |
| miR K12-3 | Sarcoma | | | | | |
| | Associated | | | | | |
| | Virus | | | | | |
| KSHV- | Kaposi | miR-K12-1 | auuacaggaaacuggguguaagc | 23 | 67 | 16 |
| miR K12-1 | Sarcoma | | | | | |
| | Associated | | | | | |
| | Virus | | | | | |
| HCMV- | Human | miR-UL22A-1 | uaacuagccuucccgugaga | 20 | 68 | 17 |
| UL22A-1 | Cytomegalo- | | | | | |
| | virus | | | | | |
| HCMV- | Human | miR-UL36-1 | ucguugaagacaccuggaaaga | 22 | 75 | 18 |
| UL36-1 | Cytomegalo- | | | | | |
| | virus | | | | | |
| HCMV- | Human | miR- | aagugacggugagauccaggcu | 22 | 67 | 19 |
| UL112-1 | Cytomegalo- | UL112-1 | | | | |
| | virus | | | | | |
| HCMV- | Human | miR- | ucguccuccccuucuucaccg | 21 | 72 | 20 |
| UL148D-1 | Cytomegalo- | UL148D-1 | | | | |
| | virus | | | | | |
| HCMV- | Human | miR-US5-1 | ugacaagccugacgagagcgu | 21 | 66 | 21 |
| US5-1 | Cytomegalo- | | | | | |
| | virus | | | | | |
| HCMV- | Human | miR-US5-2 | uuaugauaggugugacgauguc | 22 | 65 | 22 |
| US5-2 | Cytomegalo- | | | | | |
| | virus | | | | | |
| HCMV- | Human | miR-US25-1 | aaccgcucaguggcucggacc | 21 | 70 | 23 |
| US25-1 | Cytomegalo- | | | | | |
| | virus | | | | | |
| HCMV- | Human | miR-US25-2 | agcggucuguucagguggauga | 22 | 90 | 24 |
| US25-2 | Cytomegalo- | | | | | |
| | virus | | | | | |
| HCMV- | Human | miR-US33-1 | gauugugcccggaccgugggcg | 22 | 70 | 25 |
| US33-1 | Cytomegalo- | | | | | |
| | virus | | | | | |
| HIV-miR- | HIV-1 | miR-TAR-1 | ugggucucucugguuagaccag | 22 | 69 | 26 |
| TAR-1 | | | | | | |
| HIV-miR- | HIV-1 | miR-TAR-2 | cucucuggcuaacuagggaacc | 22 | 69 | 27 |
| TAR-2 | | | | | | |
| HIV-miR- | HIV-1 | miR-GAG-1 | cccuauagugcagaaccuccag | 22 | 76 | 28 |
| GAG-1 | | | | | | |
| HIV-miR- | HIV-1 | miR-GAG-2 | ccugaacuuuaaaugcauggga | 22 | 76 | 29 |
| GAG-2 | | | | | | |
| HIV-miR- | HIV-1 | miR- | uuuagggaagaucuggccuucc | 22 | 76 | 30 |
| GAG/POL-1 | | GAG/POL-1 | | | | |
| HIV-miR- | HIV-1 | miR- | gggaaggccagggaauuuucuu | 22 | 76 | 31 |
| GAG/POL-2 | | GAG/POL-2 | | | | |
| HIV-miR- | HIV-1 | miR-nef-1 | ccugagagagaaguguuagagu | 22 | 71 | 32 |
| nef-1 | | | | | | |
| HIV-miR- | HIV-1 | miR-nef-2 | cuagcauuucaucacguggccc | 22 | 71 | 33 |
| nef-2 | | | | | | |
| HIV-miR | HIV-1 | miR-LTR-1 | gggaacccacugcuuaagccuc | 22 | 75 | 34 |
| LTR-1 | | | | | | |
| HIV-miR- | HIV-1 | miR-LTR-2 | Uucaaguagugugugcccgucu | 22 | 75 | 35 |
| LTR-2 | | | | | | |

In one embodiment, the viral miRNA is any of the viral miRNAs listed in Table 1. In a preferred embodiment, the viral miRNA is abundant in the cell. In one embodiment, the viral miRNA is expressed during a lysogenic phase of the viral life cycle. In another embodiment, the viral miRNA is expressed during the lytic phase of the viral life cycle. In a preferred embodiment, the viral miRNA is expressed during the initial phases of the viral life cycle, for example, following infection of the host cell. In a more preferred embodiment, the viral miRNA is expressed during all phases of the viral life cycle.

In particular embodiments, the viral miRNA recruiting moiety may be designed to target viral miRNAs in order to induce gene silencing of viral and/or host genes. For example, the viral miRNA recruiting moiety may be designed to recruit viral miRNAs associated with any of the viruses described herein. In a particular embodiment, the viral miRNA recruiting moiety is designed to recruit miRNAs associated with HCMV, KSHV, HIV-1 or Epstein Barr (EBV). For example, the miRNA recruiting moiety may be designed to recruit an mRNA endogenous to HIV as shown in Table 1 and as disclosed in Bennasser et al. (Retrovirology (2004) 1(1):43), hereby incorporated herein by reference. Alternatively, the miRNA recruiting moiety may be designed to recruit an miRNA endogenous to Epstein Barr virus as shown in Table 1 and as disclosed in Pfeffer et al. (Science (2004) 304(5671):734-736), or as described in Cai et al., (Plos Pathog, 2(3):e23, (2006)). In other exemplary embodiments, the miRNA may be designed to recruit certain miRNAs endogenous to Kaposi's sarcoma-associated herpesvirus (KSHV) as shown in Table 1 or as depicted in Cai et al., Proc. Natl. Acad. Sci., 102(15): 5570-5575 (2006) or Samols et al., J. of Virology, 79(14): 9301-9305 (2006). In yet other exemplary embodiments, the mRNA may be designed to recruit the miRNAs endogenous to Human Cytomegalovirus (HCMV) shown in Table 1 or as described in Dunn et al., Cell Microbiol., 7(11): 1684-95 (2005).

Viral miRNA recruiting portions may be designed to recruit any naturally-occurring viral miRNA identified from publically-available and searchable databases (see Griffiths-Jones S. “The microRNA Registry”, NAR (2004) 32, Database Issue, D109-D111 or through online searching at the Sanger Institute website, both of which are hereby incorporated herein by reference). Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). Alternatively, the viral miRNA targeting portion can be designed to recruit a viral miRNA that is cloned from a virally-infected cell using methods that are known in the art, for example as described in International PCT Publication No. WO 03/029459; Elbashir et al., Genes & Dev., (2001), 15: 188). Briefly, these methods may comprise isolating total RNA from the virally-infected cell, size-fractionating the total RNA (e.g. by gel electrophoresis or gel filtration) to obtain a population of small RNAs, ligating 5′- and 3′-adapter molecules to the ends of the fractionated small RNA molecules, reverse-transcribing said adapter-ligated RNAmolecules, and characterizing said reverse transcribed RNA molecules, for example, by amplification (e.g., RT-PCR), concatamerization, cloning, and sequencing. Confirmation that a cloned miRNA is of viral, and not host, origin can be determined by examining (e.g. by BLAST alignment) the degree of sequence homology between the sequenced miRNA and the genomic DNA sequence of the virus that infected the cell and/or the genomic DNA of the host cell from which the miRNA was cloned. Viral miRNAs would be expected to have low sequence homology with all portions of the host cell genomic DNA and high sequence sequence homology (e.g. 100% homology) to a portion of the viral genomic DNA. Alternatively, the viral origins of the viral miRNA can be experimentally confirmed by detecting (e.g. by Northern blot) the presence of the viral miRNA in the infected cell and/or failing to detect expression of the viral miRNA in an uninfected cell.

In other embodiments, the viral miRNA recruiting portion may be designed to recruit a putative viral miRNA molecule, such as the viral “miRNA-like” molecules which are predicted to be derived from certain noncoding, structural viral RNAs (svRNAs) that share structural features (e.g. stem loops and bulges) with pre-miRNA. Such svRNAs, most notably the VA RNAs of the Adenovirus family, have been shown to be processed by Dicer to form miRNA-like molecules capable of mediating RNAi (see International PCT Publication WO 2005/019433, which is incorporated herein by reference). Other virus families and viruses (e.g. herpesviruses and lentiviruses) encode svRNAs. Exemplary svRNAs include VA-RNAI, VA-RNAII, EBER 1, EBER 2, MHV-68, CMER, RRE, TAR, POLADS, PAN RNA and IRES.

In other embodiments, the viral miRNA recruiting portion may be designed to recruit a siRNA which is produced in an infected cell by the processing of a longer double-stranded viral RNA precursor. Preferably, an siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs).

The viral miRNA recruiting moiety should be of sufficient size to effectively recruit the desired viral miRNA. The length of the recruiting moiety will vary greatly depending, in part, on the length of the viral miRNA and the degree of complementarity between the viral miRNA and the recruiting moiety. Generally, viral miRNAs are between about 17 to about 23 nucleotides in length. Accordingly, in various embodiments of the present invention, the viral miRNA recruiting moiety is less than about 25, 24, 23, 22, 21, 20, 19, 18, 17, 16, 15, 14, 13, 12, 11, 10, 9, 8, 7, 6, 5, 4, 3 or 2 nucleotides in length. In one embodiment, the recruiting moiety is about 13 to about 21 nucleotides in length. In another embodiment, the recruiting moiety is about 13, 14, 15 or 16 to 21 nucleotides in length. In a particular embodiment, the recruiting moiety is about 13, 14 or 15 nucleotides in length.

b) mRNA Targeting Moiety

The mRNA targeting moiety, as described above, is capable of capturing a specific target mRNA. According to the invention, expression of the target mRNA is undesirable, and, thus, RNA silencing of the target mRNA is desired. In one embodiment, the target mRNA is expressed by the virus. For example, the target mRNA may encode for a viral coat protein, necessary for the virus to infect a host cell. In other embodiments, the target mRNA is expressed by the host. For example, expression of the host mRNA may be required by the virus to facilitate a productive infection of the host.

The mRNA targeting moiety should be of sufficient size to effectively bind the target mRNA. The length of the targeting moiety will vary greatly depending, in part, on the length of the target mRNA and the degree of complementarity between the target mRNA and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a particular one embodiment, the targeting moiety is about 15 to about 25 nucleotides in length. In another embodiment, the targeting moiety is about 9, 10, 11, 12, 13 or 14 to about 24 nucleotides in length. In a particular embodiment, the targeting moiety is about 15 nucleotides in length, e.g., 15, 16, 17 or 18 nucleotides in length.

1) Targeted Viral mRNAs

In certain embodiments, the mRNA targeting moiety may be designed to target viral mRNAs (i.e. mRNAs encoded by viral genes) encoding a viral protein in order to induce RNA silencing of viral and/or host genes. For example, the mRNA targetting moiety may be designed to silence target viral miRNAs expressed by any of the viruses described herein. Viral mRNAs which may be targeted by the RNA-silencing agents of the invention include, but are not limited to, viral capsid proteins, viral envelope proteins, viral enzymes affecting interaction of the virus with the host protease (e.g. neuraminidases, endoglycosidases), viral enzymes transcribing the viral genome into RNA (e.g. DNA- and RNA-dependent RNA polymerases, double-stranded RNA transcriptases, single-stranded RNA transcriptases), enzymes adding specific terminal groups to viral mRNA (e.g. nucleotide phosphohydrolases, guanylyl transferases, RNA methylases, poly(A)polymerases), enzymes involved in copying retroviral RNA into DNA (e.g. reverse transcriptases, RNase H, polynucleotide ligases), enzymes involved in integrating viral DNA into the host chromosome (e.g. integrases), enzymes involved in processing of viral and/or host DNA or RNA (e.g. exo- and endo-deoxyribonucleases, exo- and endo-ribonucleases, tRNA aminoacylases), enzymes involved in the modification or processing of viral proteins (e.g. protein kinases, proteases), viral proteins required for modifying a host response to the virus (e.g. virokines which mimic cytokines, viroreceptor which bind host cytokines, viral complement-binding proteins), viral proteins which inhibit presentation of viral antigens by MHC class I molecules, or viral proteins which cause host cell death or lysis (e.g. viral peptide toxins).

In an exemplary embodiment, the mRNA targeting moiety may be designed to target an mRNA expressed by an HIV virus, including for example any one of the following mRNAs: mRNA encoding the HIV capsid protein gag, mRNA encoding the HIV envelope protein env (codes for CD4 receptor binding protein), pol mRNA (codes for enzymes generated by the virus such as reverse transcriptase, integrase and protease); mRNA encoding the regulatory proteins tat (codes for transactivation protein) or rev; and mRNA encoding the accessory proteins vpu (involved in virion release and mechanism for CD4 degradation), vpr, vif (viral infectivity factor), or nef (involved in the downregulation of CD4 cell-surface expression, the activation of T cells, and the stimulation of HIV infectivity).

In a preferred aspect of the invention, the viral mRNA molecule that is targeted specifies the amino acid sequence of a viral protein associated with an early stage of the viral life cycle. For example, the viral mRNA may be an mRNA which facilitates the viral DNA replication of a DNA virus or the transcription of the RNA of a RNA virus.

In other preferred embodiments, the viral mRNA transcript to be targeted may “delayed early mRNAs” or, more preferably, “immediate early mRNAs”. Immediate early viral mRNAs include mRNAs of viruses that are transcribed by host transcriptional machinery and accumulate in the cytoplasm if viral protein translation is inhibited. Delayed early mRNAs do not appear in the cytoplasm if protein translation is inhibited, but are retained as pre-mRNA precursors in the nucleus of the infected host cell. If protein translation is not inhibited, delayed early mRNAs are formed and are serve to block translation of late, major structural proteins.

2) Targeted Host mRNAs

In certain embodiments, the mRNA targeting moiety may be designed to target a host mRNA (i.e. a cellular mRNAs encoded by a host gene) encoding a host factor which is employed by the virus during any stage of its life cycle and/or is employed by the virus for host cell infection, replication, integration into the host genome, virulence, drug metabolism by the pathogen or host, replication or integration of the pathogen's genome, viral gene expression, or assembly of the next generation of pathogen. For example, the mRNA targeting moiety may be designed to target host factors required by any of the viruses described herein. Host factor mRNAs which may be targeted by the RNA-silencing agents of the invention include, but are not limited to, viral receptor proteins and other host proteins required for the entry of the virus into the host cell (for example, by receptor-mediated endocytosis), host factors required for translation of viral replicative factors (e.g. RNA helicases, translation initiation factors, and other viral RNA binding proteins), host factors required to inhibit translation of cellular proteins, host factors required for post-translational modification of viral proteins (e.g. chaperones), host factors required for intracellular localization (e.g. endosomal sorting, nuclear trafficking (e.g. nuclear import or nuclear export)) of viral transcripts or proteins, host factors involved in assembly and/or activation of viral replication or transcription complexes, host factors involved in selection and/or recruitment of viral replication or transcriptional templates (e.g. poly(A) binding proteins, nucleolin), host factors involved in preventing viral RNA turnover (e.g. tRNA nucleotidyl-transferase), host factors required for virion assembly, host factors required for virion release, as well as host virulence factors which enhance the capacity of the virus to cause disease in the host (e.g. host genes which reduce the immune response of host to virus). Host genes affecting viral pathogenesis can be identified, for example, by microarray analysis of genes which are highly and/or specifically expressed in virally-infected cells, and/or functional genomics approaches to identify host genes whose function is necessary to support viral replication (see, for example, Kushner et al., PNAS, (2003), 100(26): 15764-9; Cherry et al., Genes Dev., (2005), 19(4): 445-52).

In an exemplary embodiment, the mRNA targeting moiety may be designed to target a host mRNA which is necessary for to facilitate infection by the HIV virus, including for example mRNAs encoding any one of the following host proteins: the HIV co-receptors CD4, CCR5, and CXCR4 required for viral entry, the cyclophilin (CyPA) gene required for reverse transcription of the HIV genome, the host cell transcription factors (e.g. AP-1, NF-κβ, NF-AT, NF-IL-6, CREB, IRF, Sp1, LEF-1/TCF1α, Ets-1, USF, Cycliln T1, CDK9) and RNA polymerase II which are required for assembly, activation, and/or function of the HIV transcription complex, host proteins required for nuclear export of HIV transcripts (e.g., exportin, Sam68, Ran-GTP, Rev-interacting protein (hRIP)), and the host factors (e.g. Furin, Tsg101) required for assembly of the HIV.

In a preferred aspect of the invention, the target mRNA molecule of the invention specifies the amino acid sequence of a protein associated with an early stage of the viral life cycle, e.g. a virus receptor which facilitates entry of the pathogen into the host.

c) Linking Moiety (L)

According to the invention, the linking moiety refers to a domain, portion or region of the RNA-silencing agent which covalently joins or links the mRNA targeting moiety and the viral miRNA recruiting moiety. The linking moiety merely tethers the targeting moiety and the recruiting moiety. Accordingly, the linking moiety may be a discrete entity as known in the art, including, but not limited to, a carbon chain, a nucleotide sequence, polyethylene glycol (PEG) or a cholesterol. Alternatively, the linking moiety may be a simple phosphorus-containing moiety, such as a phosphodiester linkage, a phosphorothioate, or a methylphosphonates. In a particular embodiment, the linking moiety is a phosphodiester bond. Moreover, the linking moiety may be modified as necessary (as described below) to optimize the stability of the RNA-silencing agent.

In one embodiment, the linking moiety is a nucleotide sequence. The linking moiety may be of any length suitable both to allow the binding of the moieties to their respective target mRNA and viral miRNA, and to promote the RNA silencing of the target mRNA. In one embodiment, the linking moiety is less than about 50, 30, 25, 20, 19, 18, 17, 16, 15, 14, 13, 12, 11, 10, 9, 8, 7, 6, 5, 4, 3, or 2 nucleotides in length. In a particular embodiment, the linking moiety is about 5 to about 10 nucleotides in length. In another particular embodiment, the linking moiety is absent.

The RNA silencing agent, and each of the mRNA targeting moiety, the viral miRNA recruiting moiety and the linking moiety should be designed as necessary so as to promote effective RNA silencing. Factors to be considered when designing the agent and the respective domains include, but are not limited to, enhancing the ability of the agent to recruit both the mRNA and the viral miRNA, in addition to enchancing the overall stability and cellular uptake of the agent.

A. Sequence Complementarity

The RNA-silencing agents of the invention comprise mRNA targeting moiety and viral miRNA targeting moiety sequence portions that are “sufficiently complementary” to promote binding of target mRNA and viral miRNA, respectively.

Designing sequences in terms of size and complementarity to optimize binding to target sequences is well known in the art. The recruiting moiety and/or the targeting moiety may have 100% sequence identity to the complement of the viral miRNA and/or the complement of the target mRNA, respectively. However, 100% identity is not required. Greater than 80% sequence identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, between the targeting moiety (ie. the mRNA and/or the recruiting moiety) and the complement of the viral miRNA and/or target mRNA sequence is preferred. Conversely, recruiting moiety sequences with less than 80% identity to the complement of the portion of the respective viral miRNA and/or target mRNA sequence (i.e. at the site of complementarity) may be preferred in order to mediate silencing by translational repression. Generally, however, the sequence identity should be that which is sufficient to promote selective binding of the moieties to their respective targets. The invention, thus, has the advantage of being able to tolerate sequence variations (e.g. insertions, deletions, and single point mutations) that might be expected due to genetic mutation, strain polymorphism, or evolutionary divergence.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package.

Alternatively, the mRNA recruiting moiety and/or the viral miRNA recruiting moiety may be defined functionally as a nucleotide sequence (or oligonucleotide sequence) that is capable of hybridizing with a portion of the target mRNA and/or viral mRNA, respecitively, under preferred hybridization conditions, e.g., 400 mM NaCl, 40 mM PIPES pH 6.4, 1 mM EDTA, 50° C. or 70° C. hybridization for 12-16 hours; followed by washing. Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In another embodiment, the RNA-silencing agent can be tailored to favor a particular RNA silencing mechanism. For example, the capacity of the RNA-silencing agent to mediate translational repression by RNAi or sequence-dependent target mRNA cleavage by RNAi may be predicted by the distribution of non-identical nucleotides between the mRNA and/or the viral miRNA moiety sequences and their respective target sequences at the site of complementarity. In one embodiment, where gene silencing by translational repression is desired, at least one non-identical nucleotide may be inserted in the central portion of the complementarity site so that duplex formed by moiety sequence and the targeted sequence contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U).

i) Sequence Complementarity with Target mRNAs

The mRNA targeting moiety should include a sequence of sufficient size and of sufficient degree of complementarity to the target mRNA so as to effectively and selectively bind the target mRNA. Preferably, the mRNA targeting moiety has a sequence that is “sufficiently complementary” to a target mRNA sequence so as to facilitate posttranscriptional gene silencing by the RNA silencing agent, for example by RNAi or translational repression.

It has been observed that as the degree of sequence identity between a natural miRNA sequence and the corresponding target gene sequence is decreased, the tendency to mediate post-transcriptional gene silencing by translational repression rather than RNAi is increased. Therefore, in certain embodiments, the mRNA targeting moiety may have perfect or near perfect complementarity to the target mRNA so as to favor RNA silencing via the RNAi mechanism. In alternative embodiments, where RNA silencing by translational repression of the target gene is desired, the mRNA targeting moiety may comprise a sequence with partial complementarity to a target mRNA sequence. In certain embodiments, the mRNA targeting sequence has partial complementarity with one or more short sequences (complementarity sites) dispersed within the target mRNA (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, 6, or 10 sites) may be targeted in certain embodiments.

In certain embodiments, the complementarity site may reside in the 5′-untranslated region (5′-UTR) of the target mRNA. In other embodiments, the complementarity site may reside in the 3′-UTR of the target mRNA. In yet other embodiments, the complementarity site may reside in the open reading frame (ORF) of the target mRNA.

In another embodiment, the RNA-silencing agent contains a plurality of targeting moieties, each with sufficient complementarity to one or more sites on the target mRNA sequence. In a particular embodiment, at least two of the targeting moieties may have sufficient complementarity to the same site on the target mRNA sequence. Alternatively, the RNA-silencing agent contains a targeting moiety with complementarity to one site on a target mRNA sequence.

ii) Sequence Complementarity with Viral miRNAs

The recruiting moiety should include a region of both sufficient size and of sufficient degree of complementarity to the desired viral miRNA so as to effectively and selectively bind the desired viral miRNA. Preferably, the viral miRNA recruiting moiety has a sequence that is “sufficiently complementary” to a viral mRNA sequence so as to so as to facilitate posttranscriptional gene silencing by the RNA silencing agent, for example by RNAi or translational repression. More preferably, the viral miRNA recruiting moiety has a sequence that is sufficiently complementary to the antisense strand of the mature miRNA duplex.

In one embodiment, the RNA-silencing agent contains a recruiting moiety with sufficient complementarity to a plurality of viral miRNAs. In another embodiment, the RNA-silencing agent contains a plurality of recruiting moieties, each with sufficient complementarity to at least one viral miRNA. In a particular embodiment, at least two of the recruiting moieties may have sufficient complementarity to the same viral miRNA. Alternatively, the RNA-silencing agent contains a recruiting moiety with sufficient complementarity to one miRNA.

B. Modifications

In another embodiment of the invention, the RNA-silencing agent, any of the respective moities and, in particular, the linking moiety, are modified such that the in vivo activity of the agent is improved without compromising the agent's RNA silencing activity. The modifications can, in part, serve to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

RNA-silencing agents of the invention can be modified at the 5′ end, 3′ end, 5′ and 3′ end, and/or at internal residues, or any combination thereof. In one embodiment, the RNA-silencing agent of the invention comprises one or more (e.g., about 1, 2, 3, 4, 5, 6, 7, 8, 9, 10, or more) end modifications. Modification may be at the 5′ end or the 3′ end.

In certain embodiments, the internal residues of the RNA-silencing agents (e.g., the linking moiety) are modified. As defined herein, an “internal” nucleotide is one occurring at any position other than the 5′ end or 3′ end of a nucleic acid molecule, polynucleotide or oligonucleotide. An internal nucleotide can be within a single-stranded molecule or within either strand of a duplex or double-stranded molecule. In one embodiment, the RNA-silencing agent (preferably the linking moiety within an RNA-silencing agent) is modified by the substitution of at least one internal nucleotide. In another embodiment, the RNA-silencing agent is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the RNA-silencing agent (preferably the linking moiety within an RNA-silencing agent) is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the linking moiety within the RNA-silencing agent is modified by the substitution of all of the internal nucleotides.

Internal modifications can be, for example, sugar modifications, nucleobase modifications, backbone modifications. Alternatively, the modified RNA-silencing agent can contain mismatches or bulges. In one embodiment, the RNA-silencing agent of the invention comprises one or more (e.g., about 1, 2, 3, 4, 5, 6, 7, 8, 9, 10, or more) backbone-modified nucleotides (i.e., modifications to the phosphate sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group.

In another embodiment, the RNA-silencing agent of the invention includes sugar-modified nucleotides. Sugar-modified nucleotides can include modifications to any substituents of the sugar portion of the nucleotide, e.g. the 2′moiety of the ribose sugar in a ribonucleotide. The 2′ moiety can be, but is not limited to, H, OR, R, halo, SH, SR, NH₂, NHR, NR₂ or ON, wherein R is C₁-C₆ alkyl, alkenyl or alkynyl and halo is F, Cl, Br or I. In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine, 2′-deoxy-nucleotides and 2′-Ome nucleotides can also be used within modified RNA-silencing agents moities of the instant invention. Additional modified residues include, deoxy-abasic, inosine, N3-methyl-uridine, N6, N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby preorganizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

In another embodiment, the RNA-silencing agent (e.g., the linking moiety) of the invention comprises one or more (e.g., about 1, 2, 3, 4, 5, 6, 7, 8, 9, 10, or more) nucleobase-modified nucleotides (i.e., the nucleotides contain at least one non-naturally occurring nucleobase instead of a naturally occurring nucleobase). Bases may be modified to block the activity of adenosine deaminase. Exemplary modified nucleobases include, but are not limited to, uridine and/or cytidine modified at the 5-position (e.g., 5-(2-amino)propyl uridine, 5-fluoro-cytidine, 5-fluoro-uridine, 5-bromo-uridine, 5-iodo-uridine, and 5-methyl-cytidine), adenosine and/or guanosines modified at the 8 position (e.g., 8-bromo guanosine), deaza nucleotides (e.g., 7-deaza-adenosine), and O- and N-alkylated nucleotides (e.g., N6-methyl adenosine). Nucleobase-modified nucleotides for use in the present invention also include, but are not limited to, ribo-thymidine, 2-aminopurine, 2,6-diaminopurine, 4-thio-uridine, and 5-amino-allyl-uridine and the like. It should be noted that the above modifications may be combined.

In another embodiment, the RNA-silencing agent of the invention comprises a sequence wherein at least a portion (e.g., the mRNA targeting moiety or the miRNA recruiting moiety) contains one or more (e.g., about 1, 2, 3, 4, 5, 6, 7, 8, 9, 10, or more) mismatches with the respective target (e.g., mRNA or miRNA). In another embodiment (e.g., where at least a portion of the RNA-silencing agent is double stranded, the RNA-silencing agent of the invention comprises a bulge, for example, one or more (e.g., about 1, 2, 3, 4, 5, 6, 7, 8, 9, 10, or more) unpaired bases in one of the strands.

In another embodiment, the RNA-silencing agent of the invention comprises any combination of two or more (e.g., about 2, 3, 4, 5, 6, 7, 8, 9, 10, or more) modifications as described herein. For example, the RNA-silencing agent can comprise a combination of two sugar-modified nucleotides, wherein the sugar-modified nucleotides are 2′-fluoro modified ribonucleotides (e.g., 2′-fluoro uridine or 2′-fluoro cytidine) and 2′-deoxy ribonucleotides (e.g., 2′-deoxy adenosine or 2′-deoxy guanosine).

According to the invention, the RNA-silencing agent should be modified as necessary, in part, to improve stability, to prevent degradation in vivo (e.g., by cellular nucleases), to improve cellular uptake, to enhance target efficiency, to improve efficacy in binding (e.g., to the targets), to improve patient tolerance to the agent, and/or to reduce toxicity.

In one embodiment, the RNA-silencing agent has an mRNA targeting moiety or portion of about 25 to about 50 nucleotides in length. The targeting moiety or portion is on the 5′ end of the silencing agent. Adjacent the targeting moiety or portion is the linking moiety or portion. The linking moiety or portion is about 5 to about 10 nucleotides in length and has at least one modified nucleotide (e.g., a 2′-O-methyl nucleotide or a phosphorothiate nucleotide). On the 3′ end of the agent, adjacent the linker, is a miRNA recruiting moiety or portion which is about 5 to about 25 nucleotides in length. Optionally, the RNA-silencing agent may have additional modifications in the flanking portions or moieties of the agent.

In one embodiment, the RNA-silencing agent has an mRNA targeting moiety or portion of about 25 to about 50 nucleotides in length. The targeting moiety or portion is on the 3′ end of the silencing agent. Adjacent the targeting moiety or portion is the linking moiety or portion. The linking moiety or portion is about 5 to about 10 nucleotides in length and has at least one modified nucleotide (e.g., a 2′-O-methyl nucleotide or a phosphorothiate nucleotide). On the 5′ end of the agent, adjacent the linker, is a miRNA recruiting moiety or portion which is about 5 to about 25 nucleotides in length. Optionally, the RNA-silencing agent may have additional modifications in the flanking portions or moieties of the agent.

C. Production of RNA-Silencing Agents

RNA may be produced enzymatically or by partial/total organic synthesis, any modified nibonucleotide can be introduced by in vitro enzymatic or organic synthesis. In one embodiment, a silencing agent is prepared chemically. Methods of synthesizing RNA molecules are known in the art, in particular, the chemical synthesis methods as described in Verma and Eckstein (1998) Annul Rev. Biochem. 67:99-134.

Alternatively, the RNA-silencing agents can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck (1989) Methods Enzymol. 180:51-62). The RNA may be dried for storage or dissolved in an aqueous solution. The solution may contain buffers or salts to inhibit annealing, and/or promote stabilization of the single strands.

In another embodiment, RNA silencing agents are synthesized directly either in vivo, in situ, or in vitro. An endogenous RNA polymerase in the cell may mediate transcription of the RNA silencing agent in vivo or in situ, or a cloned RNA polymerase can be used for transcription of the RNA silencing agent in vivo or in vitro. For transcription from a transgene in vivo or an expression construct, a regulatory region (e.g., promoter, enhancer, silencer, splice donor and acceptor, polyadenylation) may be used to transcribe the RNA silencing agent (e.g. siRNA or or siRNA-like duplexes). Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses a RNA silencing agent from a recombinant construct may be produced by introducing the construct into a zygote, an embryonic stem cell, or another multipotent cell derived from the appropriate organism.

D. Constructs Encoding RNA-Silencing Agents

The invention also provides recombinant expression vectors comprising recombinant nucleic acids operatively linked to an expression control sequence, wherein expression, i.e. the transcription and optionally futher processing, results in one or more RNA-silencing agents or a precursor molecules thereof. The vector is preferably a DNA vector, e.g. a viral vector or plasmid, particularly an expression vector suitable for nucleic acid expression in eukaryotic, more particularly mammalian cells. The recombinant nucleic acid contained in aid vector may be a sequence which results in the transcription of the RNA-silencing agent as such, a precursor or primary transcript thereof, which may be further processed to give the RNA-silencing agent. The vector can be administered in vivo to thereby initiate RNAi therapeutically or prophylactically by expression of one or more copies of the RNA-silencing agent. Use of vectors may be advantageous because the vectors can be more stable than oligonucleotides and thus effect long-term expression of the siRNAs.

Vectors may be designed for delivery of multiple RNA-silencing agents capable of silencing multiple target mRNAs within the infected cell. Accordingly, in one embodiment, a vector is contemplated that expresses a plurality of RNA-silencing agents to decrease the likelihood that a virus may acquire resistance to a particular RNA-silencing agent. In one embodiment, a first RNA-silencing agent capable of silencing a viral target mRNA and a second RNA-silencing agent capable of silencing a host target mRNA are both encoded by a vector. In one embodiment, the vector encodes about 3 RNA silencing agents, more preferably about 5 RNA silencing agents.

In one embodiment, expression of the RNA silencing agent is driven by a RNA polymerase III (pol III) promoter (T. R. Brummelkamp et al. Science (2002) 296:550-553; P. J. Paddison et al., Genes Dev. (2002) 16:948-958). Pol III promoters are advantageous because their transcripts are not necessarily post-transcriptionally modified, and because they are highly active when introduced in mammalian cells. In another embodiment, expression of the RNA silencing agent is driven by a RNA polymerase II (pol II) promoter. Polymerase II (pol II) promoters may offer advantages to pol III promoters, including being more easily incorporated into viral expression vectors, such as retroviral and adeno-associated viral vectors, and the existence of inducible and tissue specific pol II dependent promoters.

E. Methods of Introducing RNAs and Vectors into Host Cells

Physical methods of introducing the agents of the present invention (e.g., RNA silencing agents, vectors, or transgenes) include injection of a solution containing the agent, bombardment by particles covered by the agent, soaking the cell or organism in a solution of the agent, or electroporation of cell membranes in the presence of the agent. A viral construct packaged into a viral particle would accomplish both efficient introduction of an expression construct into the cell and transcription of RNA, including RNA silencing agents, encoded by the expression construct. Other methods known in the art for introducing nucleic acids to cells may be used, such as lipid-mediated carrier transport, chemical-mediated transport, such as calcium phosphate, and the like. Thus the RNA silencing agent may be introduced along with components that perform one or more of the following activities: enhance uptake by the cell, inhibit annealing of single strands, stabilize the single strands, or otherwise increase inhibition of the target gene.

The agents may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the agent may be introduced.

Cells may be infected with a virus upon delivery of the agent or exposed to the virus after delivery of agent. The cells may be derived from or contained in any organism. The cell may be from the germ line, somatic, totipotent or pluripotent, dividing or non-dividing, parenchyma or epithelium, immortalized or transformed, or the like. The cell may be a stem cell, e.g., a hematopoietic stem cell, or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. Preferably, the cell is permissive host for the virus. For example, wherein the virus is HIV, a permissive host cell is a lymphocyte (such as a T lymphocyte), a macrophage (such as a monocytic macrophage), a monocyte, or is a precursor to either of these cells, such as a hematopoietic stem cell.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of viral protein, RNA, and/or DNA. Specificity refers to the ability to inhibit the target gene without manifesting effects on other genes, particularly those of the host cell. The consequences of inhibition can be confirmed by examination of the outward properties of the cell or organism or by biochemical techniques such as RNA solution hybridization, nuclease protection, Northern hybridization, reverse transcription gene expression monitoring with a microarray, antibody binding, enzyme linked immunosorbent assay (ELISA), integration assay, Western blotting, radioimmunoassay (RIA), other immunoassays, and fluorescence activated cell analysis (FACS).

For RNA silencing in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of siRNA may result in inhibition in a smaller fraction of cells (e.g., at least 10%, 20%, 50%, 75%, 90%, or 95% of targeted cells).

Quantification of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory double-stranded RNA, or translated polypeptide may be detected with an antibody raised against the polypeptide sequence of that region.

The RNA silencing agent may be introduced in an amount that allows delivery of at least one copy per cell. Higher doses (e.g., at least 5, 10, 100, 500 or 1000 copies per cell) of material may yield more effective inhibition; lower doses may also be useful for specific applications.

Methods of Treatment

The present invention further provides for both prophylactic and therapeutic methods for treating a subject (e.g., a human) having or at risk of (or susceptible to) infection with a virus (e.g., HIV virus or EBV virus). The prophylactic and therapeutic methods of the invention involve administering therapeutic compositions comprising RNA silencing agents or vectors or transgenes encoding said agents. In preferred embodiments, the RNA silencing agent is capable of binding to a viral miRNA that is expressed by a virus infecting the subject.

In certain embodiments, the RNA silencing agents of the invention can be used to treat viral infections or diseases or disorders associated with viruses. The viral disease may be characterized, caused by, or associated with the overexpression or overactivity of a host or viral protein. Accordingly, administration of an RNA-silencing agent that has an mRNA targeting moiety capable of binding the mRNA encoding the overexpressed or overactive protein, can mediate post-transcriptional silencing said mRNA.

In another embodiment, the RNA silencing agents of the invention can be used to prevent propogation of a virus. Indeed, viruses encode endogenous miRNAs that may affect, for example, expression of endogenous host genes. Accordingly, the RNA silencing agents of the invention can be designed to direct viral miRNAs to silence viral gene targets, for example, in order to treat a viral infection, to prevent viral replication, and/or to prevent the propagation of the virus. In particular embodiments, the RNA silencing agents of the present invention may be designed to recruit viral miRNAs endogenous to any of the viruses described herein, and in particular, HIV or Epstein Barr viruses. RNA silencing agents used in this manner exhibit particular target specificity in that the RNA silencing agents will target only those cells which have been infected by the targeted virus.

In certain embodiments, the RNA silencing agents of the invention can be used to identify and/or validate potential targets for therapeutic interventions against viral infections or diseases or disorders association with viral infections, for example, AIDS. The RNA silencing agents of the invention can be used for target identification and/or validation animal models or, alternatively, in appropriate cell culture models. Animal models include, but are not limited to, mammalian models, for example, non-human primate models (e.g. ape, monkey or baboon models) and rodent models (e.g., mouse or rat models), as well as non-mammalian biological systems, for example, Drosophila systems, C. elegans and the like. Cell culture models feature, for example human primary cells, human cell lines (e.g. HeLa, Detroit-6, Minnesota-EE, L-132, Intestine 407, Chang liver KB, Detroit 98, AV3, Hep-2, J-111, WISH), non-human primate (e.g. monkey) cell lines (e.g. LLC-MK2, BS-C-1), rodent (e.g. mouse, hamster, rate) cell lines (e.g. HaK, BHK, Don, CHO, L, 929, 2472, 2555, S-180, 3T3), or chicken embryos (e.g. chicken eggs). Preferably said animal or cell culture models are permissive hosts for productive infection and/or replication by the virus of interest. Target validation methods of the invention involve, for example, administering a RNA silencing agent of the invention to an infected cell or organism comprising a potential therapeutic target mRNA and determining the effect of the silencing agent on the ability of virus to infect other, uninfected cells. Alternatively, the RNA silencing can be administered to an un-infected cell or organism comprising a potential therapeutic target mRNA and determining the ability of the silencing agent to infect the cell.

The RNA silencing agents of the invention can be also tested in an appropriate animal model. For example, an RNA-silencing agent as described herein can be used in an animal model to determine the efficacy, toxicity, or side effects of treatment with said agent.

In one embodiment, a target mRNA is potentially expressed as a viral mRNA which is necessary for viral uptake, viral gene expression (e.g. transcription of viral genes, translation of viral proteins), virion assembly, drug resistance, and or virulence factors such as factors influencing host cell growth, host cell proliferation, host cell apoptosis, host cell morphology, host cell differentiation, host cell migration, host signal transduction, host cell cycle regulation, host morphogenesis, host biosynthesis of cellular factors, or host resistance mechanisms to viral infection.

In another embodiment, the target mRNA is a host mRNA involved in or associate with a stage of the viral life cycle, including but not limited to viral receptor proteins and other host proteins required for the entry of the virus into the host cell, host factors required for translation and/or transcription of viral replicative factors (e.g. RNA helicases and other viral RNA binding proteins (e.g. La, PTB), ribosomal proteins (e.g., S1, HF1), translation initiation or elongation factors (e.g. eIF3, EF-Tu, EF-Ts)), host factors required to inhibit translation of cellular proteins, host factors required for post-translational modification of viral proteins (e.g. chaperones), host factors required for intracellular localization (e.g. endosomal sorting, nuclear trafficking) of viral proteins (e.g., tubulin, actin, chaperones), host factors involved in assembly and/or activation of viral replication or transcription complexes (e.g. host transcription factors, host RNA- or DNA-polymerases), host factors involved in selection and/or recruitment of viral replication or transcriptional templates (e.g. poly(A) binding proteins, nucleolin), host factors involved in preventing viral RNA turnover (e.g. tRNA nucleotidyl-transferase), host factors required for virion assembly, host factors required for virion release, as well as host virulence factors which enhance the capacity of the virus to cause disease in the host (e.g. host genes which reduce the immune response of host to virus). A RNA silencing agent specific for the target is administered to an appropriate cell or animal model under conditions sufficient for silencing of the target and the effect of the silencing agent on the process is determined.

In another embodiment, a target is potentially involved in a disease or disorder or other pathological condition and the RNA silencing agent specific for the target is administered to an appropriate cell or animal model under conditions sufficient for silencing of the target and the effect of the silencing agent on the disease or disorder or other pathological condition is determined. The effect of the silencing agent can be determined as a direct effect on expression or activity of the target or the expression or activity of a downstream molecule or process effected or regulated by said target. The effect of the silencing agent can be determined as its effect on a process regulated by or associated with said target. The effect of the silencing agent can be determined as an effect on a biological characteristic or phenotype associated with said target. In appropriate animal models, for example, in animal models of disease or disorder, the effect of the silencing agent can be determined as an improvement, reversal, or attenuation is the disease or disorder or one or more symptoms or biological features of the disease or disorder.

The compositions and methods of the present invention can serve to validate particular targets for further study, for example, ultimately for the treatment of a disease or disorder. For example, using the techniques of the present invention, the effects of the repression of particular genes on cellular function may be analyzed.

In achieving a therapeutic or prophylactic effect, the compositions and methods of the present invention have the added advantage of inducing RNA silencing only in those cells that are infected with the virus expressing the miRNA for which the RNA silencing agent is designed to recruit. Accordingly, the RNA silencing agent may be freely administered with the knowledge that undesirable RNA silencing will not occur in non-targeted cells (e.g. uninfected cells), thereby providing a tissue specificity for the compositions and methods of the present invention.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the RNA-silencing agents of the present invention according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a viral infection or a disease or condition associated with viral infection (e.g. AIDS associated with HIV infection), by administering to the subject a prophylactically effective agent that includes any of the RNA-silencing agents or vectors or transgenes discussed herein. Administration of a prophylactic agent can occur prior to the manifestation of symptoms characteristic of a viral infection, such that the associated disease or disorder is prevented or, alternatively, delayed in its progression. Subjects at risk for a disease which is caused or contributed to by viral infection can be identified by, for example, any or a combination of diagnostic or prognostic assays as described herein.

In a preferred embodiment, the prophylactically effective agent is administered to the subject prior to exposure to the virus to prevent its entry into the host's cells. In another embodiment, the agent is administered to the subject after exposure to the virus to delay or inhibit its progression, or prevent its entry or replication in healthy cells or cells that do not contain a virus. Thus, the method is prophylactic in the sense that healthy cells are protected from viral infection. The methods generally include administering the agent to the subject such that viral replication or infection is prevented or inhibited. Preferably, viral entry is inhibited or prevented. Additionally or alternatively, it is preferable that viral replication is inhibited or prevented. In one embodiment, the RNA silencing agent induces RNA silencing of a viral or host mRNA involved in an early stage of the viral life cycle, for example, immediately upon entry into the cell. In this manner, the agent can prevent healthy cells in a subject from becoming infected. In another embodiment, the RNA silencing agent is a viral or host mRNA involved a late stage of the viral life cycle. Any of the strategies discussed herein can be employed in these methods, such as administration of a vector that expresses a plurality of RNA silencing agents sufficiently complementary to the viral genome to mediate RNA silencing. Any of the strategies discussed herein can be employed in these methods, such as administration of an RNA silencing agent capable of targeting an exon present in a viral mRNA that is translated into more than one protein, e.g., an RNA silencing agent that targets an exon or UTR shared by a two or more viral mRNAs or an exon or UTR of a single mRNA that expresses a viral protein precursor that is subsequently cleaved to produce two or more viral proteins. Additionally or alternatively, a vector that expresses a plurality of RNA silencing agents sufficiently complementary to the viral mRNA can be employed.

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect analysis of appropriate patient samples (e.g., blood and/or tissues).

B. Therapeutic Methods

Another aspect of the invention pertains to methods of modulating target gene expression, protein expression or activity for therapeutic purposes. Accordingly, in an exemplary embodiment, the modulatory method of the invention involves contacting a cell capable of expressing a target gene with a therapeutic agent (e.g., an RNA-silencing agent) that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which the target gene is abnormally unregulated and/or in which decreased target gene activity is likely to have a beneficial effect.

Another aspect of the invention pertains to methods of modulating target gene expression, protein expression or activity for therapeutic purposes. Accordingly, in an exemplary embodiment, the modulatory method of the invention involves contacting a cell infected with the virus with a therapeutic agent (e.g., a RNA silencing agent or vector or transgene encoding same) that is specific for a portion of the virus or host genome such that RNA silencing is mediated. These modulatory methods can be performed ex vivo (e.g., by culturing the cell with the agent) or, alternatively, in vivo (e.g., by administering the agent to a subject). The methods can be performed ex vivo and then the products introduced to a subject (e.g., gene therapy).

The therapeutic methods of the invention generally include initiating RNA silencing by administering the RNA silencing agent or a vector or transgene encoding said agent to a subject infected with the virus. In preferred embodiment, the virus expresses a viral miRNA targeted by said agent. The subject can be administered one or more RNA silencing agents, or vectors that express one or more RNA silencing agents, or transgenes that encode one or more RNA silencing agents. The therapeutic methods of the invention are capable of reducing viral production (e.g., viral titer), by about 30-50-fold, preferably by about 60-80-fold, and more preferably about (or at least) 90-fold, 100-fold, 200-fold, 300-fold, 400-fold, 500-fold or 1000-fold.

In a preferred embodiment, infected cells are obtained from a subject and analyzed to determine one or more sequences from the virus and/or host genomes present in that subject (e.g. one or more viral miRNAs or precursor sequences encoding said viral miRNAs, one or more target viral mRNA sequences or viral genes encoding said sequence, one or more target host mRNA sequences or host genes encoding said sequences). RNA silencing agents are then synthesized to be sufficiently homologous to bind to both a viral miRNA and a host or viral target mRNA present in the subject (or vectors are synthesized to express such RNA silencing agnet), and delivered to the subject to mediate RNA silencing. This approach is advantageous because it addresses the particular virus or host mutations present in the subject. This method can be repeated periodically, to address further mutations in that subject and/or provide boosters for that subject.

C. Combined Prophylactic and Therapeutic Methods

The therapeutic or prophylactic agents and methods of the present invention can be used in co-therapy with other anti-viral approaches. For example, the prophylactic or therapeutic pharmaceutical compositions of the present invention can contain other pharmaceuticals, in conjunction with a vector according to the invention, when used to therapeutically treat viral infections. These other pharmaceuticals can be used in their traditional fashion (i.e., as agents to treat infection), as well as more particularly, in the method of selecting for conditionally replicating viruses in vivo. Representative examples of these additional pharmaceuticals that can be used in combination with the agents of the invention, include antiviral compounds, immunomodulators, immunostimulants, antibiotics, and other agents and treatment regimes (including those recognized as alternative medicine). Antiviral compounds include, but are not limited to, ddI, ddC, zidovudine, ddI, ddA, gancylclovir, fluorinated dideoxynucleotides, nonnucleoside analog compounds such as nevirapine (Shih, et al., PNAS 88: 9978-9882 (1991)), TIBO derivatives such as R82913 (White, et al., Antiviral Research 16: 257-266 (1991)), and BI-RJ-70 (Shih, et al., Am. J. Med. 90 (Suppl. 4A): 8S-17S (1991)). Immunomodulators and immunostimulants include, but are not limited to, various interleukins, CD4, cytokines, antibody preparations, blood transfusions, and cell transfusions.

When given in combined therapy, the other antiviral compound, e.g., can be given at the same time as a vector according to the invention, or the dosing can be staggered as desired. The vector also can be combined in a composition. Doses of each can be less, when used in combination, than when either is used alone.

A RNA-silencing agent or vector encoding said agent according to the invention can be delivered to cells cultured ex vivo prior to reinfusion of the transfected cells into the patient or in a delivery vehicle complex by direct in vivo injection into the patient or in a body area rich in the target cells. The in vivo injection may be made subcutaneously, intravenously, intramuscularly or intraperitoneally. Techniques for ex vivo and in vivo gene therapy are known to those skilled in the art. Generally, the compositions are administered in a manner compatible with the dosage formulation, and in such amount as will be prophylactically and/or therapeutically effective. The quantity to be administered depends on the subject to be treated, including, e.g., whether the subject has been exposed to virus or infected with virus, or is afflicted with a viral disease or disorder, and the degree of protection desired. Suitable regimens for initial administration and booster shots are also variable but are typified by an initial administration followed by subsequent inoculations or other administrations. Precise amounts of active ingredients required to be administered depend on the judgment of the practitioner and may be peculiar to each subject. It will be apparent to those of skill in the art that the therapeutically effective amount of a composition of this invention will depend upon the administration schedule, the unit dose of agent (e.g., RNA silencing agent, vector and/or transgene) administered or expressed by an expression plasmid that is administered, whether the compositions are administered in combination with other therapeutic agents, the immune status and health of the recipient, and the therapeutic activity of the particular nucleic acid molecule, delivery complex, or ex vivo transfected cell.

D. Disease Indications

In one embodiment, the present invention provides methods for the treatment or prevention of diseases associated with viral infection (e.g. virally-transmitted diseases) using the RNA-silencing agents disclosed herein. Diseases associated with viral infection include any diseases or disorders caused by viral infection, or diseases or disorders where susceptibility to viral infection is a symptom or characteristic of the disease (e.g., immune disorders such as AIDS). Molecules of the invention are engineered as described herein to target expressed sequences of a virus, thus ameliorating viral activity and replication. The molecules can be used in the treatment and/or diagnosis of viral infected tissue. Also, such molecules can be used in the treatment of virus-associated carcinomas, such as hepatocellular cancer.

Diseases or disorders associated with poxvirus infections or symptoms thereof include smallpox, cowpox, tanapox, yabapox, contagious postular dermatitis, eczema, eethyma, Milker's nodule infections, Molluscum contagiosum, and other skin and mucous membrane lesions.

Diseases or disorders associated with herpesvirus simplex infections or symptoms thereof include eczema herpeticum, herpesviral vesicular dermatitis, gingivostomatitis, pharyngotonsillitis, herpesviral meningitis, herpesviral encephalitis, herpesviral ocular disease, disseminated herpesviral disease, infection of the genitalia and reproductive tract, infection of the perianal skin and rectum, and oral infections.

Diseases or disorders associated with varicellovirus infections or symptoms thereof include varicella meningitis, varicella encephalitis, varicella pneumonia, zoster meningitis, zoster encephalitis, zoster ocular disease, shingles, chickenpox.

Diseases or disorders associated with cytomegalovirus infections or symptoms thereof include mononucleosis, pneumonitis, hepatitis, and pancreatitis.

Diseases or disorders associated with lymphocryptovirus infections or symptoms thereof include Epstein-Barr disease, mononucleosis, Hodgkin's disease, pneumonia, Burkitt's lymphoma.

Diseases or disorders associated with roseolovirus infections or symptoms thereof include roseola infantum, exanthema subitum, sixth disease, and 3 day fever exanthema.

Diseases or disorders associated with rhadinovirus infections or symptoms thereof include Kaposi's sarcoma and other sarcomas, eczema herpaticum.

Diseases or disorders associated with adenovirus infections or symptoms thereof include adenoviral pneumonia, adenoviral encephalitis, adenoviral meningitis, adenoviral enteritis, keratoconjunctivitis, infantile diarrhea, pharyngeal conjunctivitis, lower respiratory tract infection, and persistent infection of the kidney.

Diseases or disorders associated with papillomavirus infections or symptoms thereof include papilloma, viral warts, and neoplasms of the bladder, cervix, and larynx.

Diseases or disorders associated with parvovirus infections or symptoms thereof include rubella, erethyma infectiosum, pediatric exanthema, and haemolytic crisis in people with sickle cell anemia.

Diseases or disorders associated with hepadnovirus infections or symptoms thereof include acute hepatitis, chronic hepatitis, liver cirrhosis, primary hepatocellular carcinoma, and hepatic coma.

Diseases or disorders associated with cytomegalovirus infections or symptoms thereof include mononucleosis, pneumonitis, hepatitis, and pancreatitis.

Diseases or disorders associated with retrovirus infections or symptoms thereof include immune deficiency syndromes (e.g. AIDS), opportunistic infections (e.g. parasitic infections), slim disease, encephalopathy, lymphopathy, and acute HIV infection syndrome.

Diseases or disorders associated with reovirus infections or symptoms thereof include enteritis, gastroenteritis, and diarrhea.

Diseases or disorders associated with filovirus infections or symptoms thereof include Ebola disease, Marburg disease, and hemorrhagic fevers.

Diseases or disorders associated with respirovirus infections or symptoms thereof include pneumonia and respiratory tract infections (e.g. acute bronchitis).

Diseases or disorders associated with rubulavirus infections or symptoms thereof include mumps, orchitis, meningitis, encephalitis, and pancreatitis.

Diseases or disorders associated with mrobillivirus infections or symptoms thereof include measles, subacute scleorising subencephalitis, meningitis, encephalitis, pneumonia, otitis media, and persistent infections.

Diseases or disorders associated with pneumovirus infections or symptoms thereof include respiratory syncitial virus pneumonia and acute bronchitis.

Diseases or disorders associated with rhabdovirus infections or symptoms thereof include rabies, encephalitis, and fever.

Diseases or disorders associated with orthomyxovirus infections or symptoms thereof include the common cold, pneumonia, and other respiratory diseases.

Diseases or disorders associated with bunyavirus infections or symptoms thereof include the hemorrhagic fever and other acute fevers, pulmonary syndrome, renal syndrome, acute respiratory distress syndrome, and encephalitis.

Diseases or disorders associated with orthomyxovirus infections or symptoms thereof include the common cold, pneumonia, and other respiratory diseases.

Diseases or disorders associated with coronavirus infections or symptoms thereof include SARS, common cold, and gastrointestinal infections.

Diseases or disorders associated with picornavirus infections or symptoms thereof include vesicular pharyngitis, vesicular stomatitis, encephalitis, meningitis, viral enteritis, bronchitis, polio myelitis, paralysis, and diarrhea.

Diseases or disorders associated with enterovirus infections or symptoms thereof include vesicular pharyngitis, vesicular stomatitis, encephalitis, meningitis, viral enteritis, bronchitis, polio myelitis, paralysis, and diarrhea.

Diseases or disorders associated with rhinovirus infections or symptoms thereof include the common cold, upper respiratory tract infection, and acute bronchitis.

Diseases or disorders associated with hepatovirus infections or symptoms thereof include Hepatitis A, hepatitis, and diarrhea.

Diseases or disorders associated with calicivirus infections or symptoms thereof include acute gastroenteritis and acute gastroenteropathy.

Diseases or disorders associated with togavirus infections or symptoms thereof include febrile illness, sever chills anthralgia, leucopoenia, rash, viral polyarthritis and rush, and severe encephalitis.

Diseases or disorders associated with flavivirus infections or symptoms thereof include Japanese encephalitis, West Nile fever, Dengue fever, Yellow fever, and hemorrhagic fever.

Diseases or disorders associated with hepacivirus infections or symptoms thereof include Hepatitis C, acute hepatitis, and chronic hepatitis.

Pharmaceutical Compositions

The invention pertains to uses of the above-described RNA-silencing agents for therapeutic treatments as described infra. Accordingly, the RNA-silencing agents of the present invention can be incorporated into pharmaceutical compositions suitable for administration. Such compositions typically comprise the RNA-silencing agent or other modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In various embodiments, the pharmaceutical composition of the present invention includes an RNA-silencing agent and an agent suitable for delivery to a subject. Alternatively, the invention includes an RNA-silencing agent conjugated to an agent suitable for delivery to a subject. Suitable delivery agents include, but are not limited to, proteinaceous agents (e.g., peptides), hydrophobic agents or lipid-based agents.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al, Nature 418:38-39, 2002 (hydrodynamic transfection); Xia et al, Nature Biotechnol, 20:1006-1010, 2002 (viral-mediated delivery); or Putnam, Am. J. Health Syst. Pharm. 53:151-160, 1996, erratum at Am. J. Health Syst. Pharm. 53:325, 1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable mtcroparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

A therapeutically effective amount of a composition containing a compound of the invention (e.g., an RNA-silencing agent) (i.e., an effective dosage) is an amount that inhibits expression of the polypeptide encoded by the target gene by at least 30 percent. Higher percentages of inhibition, e.g., 45, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. The compositions can be administered one time per week for between about 1 to 10 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments.

It is furthermore understood that appropriate doses of a composition depend upon the potency of composition with respect to the expression or activity to be modulated. When one or more of these molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into expression constructs, e.g., viral vectors, retro viral vectors, expression cassettes, or plasmid viral vectors, e.g., using methods known in the art, including but not limited to those described in Xia et al., (2002), supra. Expression constructs can be delivered to a subject by, for example, inhalation, orally, intravenous injection, local administration (see U.S. Pat. No. 5,328,470) or by stereotactic injection (see, e.g., Chen et al (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration

This invention is further illustrated by the following examples which should not be construed as limiting. The contents of all references, patents and published patent applications cited throughout this application are incorporated herein by reference.

Exemplification

The following examples describe inducing gene silencing in cells by targeting viral miRNA (e.g. HIV miRNA) and RISC to an HIV or host mRNA.

### EXAMPLE 1

### Probing HIV miRNA as an Effector in RNA Silencing

In the instant example, a synthetic HIV miRNA is recruited to a target mRNA using a 2′-O-methyl oligonucleotide complementary to both the HIV miRNA and the mRNA target. 2′-O-methyl oligonucleotides have been shown to be irreversible, stoichiometric inhibitors of miRNA function (Hutvagner et al. (2004) PLOS Biology, in press). The method recruits the viral miRNA-programmed RISC to the target mRNA to prevent translation of the target mRNA.

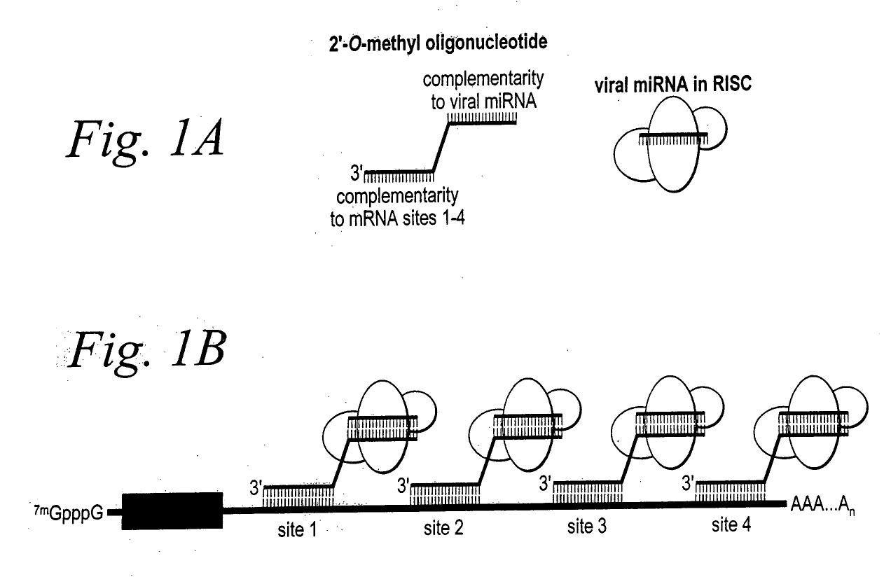

FIG. 1 depicts interactions between the designed 2′-O-methyl oligonucleotide and a viral miRNA. FIG. 1 further depicts the general design of an embodiment of the 2′-O-methyl oligonucleotide appropriate for the present example. The 3′ end of the oligonucleotide is designed to bind to an mRNA. The 5′ end of the oligonucleotide is complementary to the sequence of a viral miRNA, in this case HIV-miR-GAG/POL-1 or HIV-miR-GAG/POL-2. The diagram shows four sites of oligonucleotide complementarity in the 3′UTR of an mRNA encoding the luciferase reporter protein. Four sites are shown to be more effective than one to three sites for translational repression of the luciferase reporter mRNA. The gray spheres depict RISC proteins associated with the viral miRNA.

For the present example, 2′-O-methyl oligonucleotides are synthesized with two functional domains: an oligonucleotide region complementary to a sequence of a luciferase reporter mRNA expressed by the cell and a domain complementary to HIV-miR-GAG/POL-1 or HIV-miR-GAG/POL-2 miRNA. Ongoing studies are expected to disclose additional viral miRNAs expressed by HIV. Results from these studies will enable testing of several different HIV miRNA constructs.

Three tests are performed. In the first, a series of 2′-O-methyl oligonucleotides with different lengths of complementary sequence in each domain (e.g. 24, 21, 18, 15, or 12 nucleotides) are synthesized to determine the minimal sequence required for effective silencing of the reporter mRNA. The target luciferase mRNA is engineered to have multiple sites for oligonucleotide complementation, so that the proximal 5′ part of the oligonucleotide binds to these multiple identical 21 nucleotide ‘sites’ in series. In the second, a series of 2′-O-methyl oligonucleotides with complementarity to different portions of the target luciferase mRNA sequence (e.g. 5′-UTR, ORF, 3′UTR) are synthesized to determine which portion of the target sequence is most effectively targeted. In the third, a series of oligonucleotides with different chemical modifications (e.g. 2′-O-methyl, Locked Nucleic Acids (LNAs)) are synthesized to determine which chemical modification is most effective or potent in gene silencing.

In each test, synthetic viral miRNAs and the oligonucleotide constructs are co-transfected into human (e.g. HeLa) cells with a cationic transfection agent. Because the oligonucleotides contain sequence fully complementary to the viral miRNA, the oligonucleotide is proposed to attract RISC only in those cells which have been successfully co-transfected with synthetic viral miRNA. The oligonucleotide lacks modifications necessary to attract RISC without binding miRNA (5′ phosphate, 3′-OH, nucleotide overhangs). Subsequently, the cell is co-transfected with plasmid encoding the targeted Renilla luciferase mRNA and a plasmid enconding a non-targetted, firefly luciferase reporter mRNA which serves as an internal control. After 24 hours, cells are harvested to test for the activity of the Renilla and control luciferases by standard assays. Gene silencing of the luciferase reporter is measured by luciferase activity in a luminometer. The activity of Renilla luciferase is normalized to that of the firefly luciferase.

Analysis. Controls include (1) transfection of luciferase cDNA with an oligonucleotide that lacks sequence with complementary to the target mRNA; (2) transfection of luciferase cDNA without oligonucleotides to show basal luciferase reporter activity and (3) transfection of luciferase cDNA plus oligonucleotide without HIV miRNA. Differences in luciferase reporter activities are compared with ANOVA and Bonferroni correction, to establish significance (p<0.05). At least three separate tests are carried out. 2′-O-methyl oligonucleotides which are most effective in silencing luciferase activity are selected for further modification (e.g. chemical modification with Locked Nucleic Acids (LNAs)) and testing to determine if the efficiency or potency of gene silencing can be enhanced.

### EXAMPLE 2

### Recruiting Expressed HIV miRNAs in an HIV Infected Host Cell for Gene Silencing

In the instant example, a viral miRNA expressed in HIV infected cells is recruited to effect silencing of an mRNA that is essential for HIV infection or replication. The method employs oligonucleotides comprising sequences that are complementary to both an HIV miRNA and an mRNA target sequence expressed by the host cell or HIV.

For the present example, oligonucleotides are synthesized with two functional domains: a domain complementary to HIV-miR-GAG/POL-1 or HIV-miR-GAG/POL-2 miRNA, and an oligonucleotide region complementary to an mRNA sequence expressed by the virus (e.g. HIV protease) or the infected cell (e.g. the host cell chemokine receptor CCR5). Oligonucleotides can be designed to test silencing of any mRNA encoded by the HIV genome or any mRNA required by the HIV virus during its replication cycle.

In the present example, each oligonucleotide is transfected into CD4+ human astroglioma U87 cells which are stably co-transfected with CCR5 and CXCR4 (see Princen et al., Retrovirology, (2004), 1:2) and previously infected with a laboratory strain of HIV-1 (e.g., the T-Tropic (X4) HIV-1 molecular clone NL4.3, National Institute of Allergy and Infectious Disease AIDS Reagent program, Bethesda, Md.). The effectiveness of the oligonucleotide in silencing the target mRNA sequence (in this case, CCR5 or pro mRNA) is determined by quantifying the amount of protein encoded by the target mRNA using a Western blot. Controls include transfection of oligonucleotide against luciferase (absent in these cells). Silencing of CCR5 or Pro protein expression measured in Western blots is compared to expression endogenous a-tubulin on LAS3000 (Fuji). The same controls and statistical analysis as used in Example 1 are applied here. Tests are repeated at least 3 times for analysis. The above experimental design may be repeated in cells which are transfected with the construct encoding a GFP fusion of the target mRNA.

### EXAMPLE 3

### Effectiveness of a Dual-Functional Oligonucleotide in Inhibiting HIV Infection of Human Cells

In the instant example, dual-functional oligonucleotides are tested for their effectiveness in inhibiting the infection of human cells by HIV, thereby reducing the viral load of the infected cell. The dual-functional oligonucleotides are complementary to an HIV miRNA (e.g. HIV-miR-GAG/POL-1 or HIV-miR-GAG/POL-2 miRNA) and a host cell mRNA (e.g. CCR5) necessary for the entry of the virus into the host cell.

In the present example, CD4+ human astroglioma U87 cells are stably co-transfected with CCR5 and CXCR4, washed, and resuspended at 5×10⁴ cells/ml in medium and seeded out in 24 well plates (see Princen et al., Retrovirology, (2004), 1:2). Cells are infected with a low concentration (e.g. 1-10 pg/ml) of a laboratory strain of HIV-1 (e.g., the T-Tropic (X4) HIV-1 molecular clone NL4.3, National Institute of Allergy and Infectious Disease AIDS Reagent program, Bethesda, Md.). The pre-infected cells are transfected with the dual-functional oligonucleotide and subsequently exposed to a high concentration (e.g. 100-1000 pg/ml) of the same HIV strain. The cytopathic effect (syncytium or giant cell formation) is evaluated microscopically at 5 days after infection.

### EXAMPLE 4

### Effectiveness of a Dual-Functional Oligonucleotide in Inhibiting HIV Production in Human Cells

In the instant example, dual-functional oligonucleotides are tested for their effectiveness in inhibiting the production of HIV virions in HIV infected cells, thereby reducing the viral load of the infected cell. In this case, the dual-functional oligonucleotides are complementary to an HIV miRNA (e.g. HIV-miR-GAG/POL-1 or HIV-miR-GAG/POL-2 miRNA) and an HIV miRNA (e.g. HIV pol mRNA) encoding a protein expressed late in the life cycle of the virus (e.g. HIV protease).

Dual-functional oligonucleotides are co-transfected with an HIV-1 molecular clone (HIV_{NL-GFP}; Welker, R., et al., J. Virol. (1998) 72, 8833-8840) into CD4-positive HeLa (Magi) cells (Kimpton, J. & Emerman, M., J. Virol. 66, 2232-2239 (1992)). Transfection of cells with an infectious molecular HIV-1 clone recapitulates late events in the viral life cycle, including production of viral RNAs, translation of viral proteins and release of virions.

To determine the level of HIV virus production, viral p24 (capsid) is protein measured at 24 hours post-transfection by an enzyme-linked immunosorbent assay (ELISA) according to a manufacturer's protocol (Beckman-Coulter). Cells transfected with dual functional oligonucleotides are compared with control experiments in which the cells not transfected with the dual functional oligonucleotide.

### EXAMPLE 5

### Effectiveness of a Dual-Functional Oligonucleotide in Inhibiting AIDS

With respect to determining the effective level in a patient for treatment of AIDS or AIDS-like disease, in particular, suitable animal models are available and have been widely implemented for evaluating the in vivo efficacy against HIV of various gene therapy protocols (Sarver, et al., AIDS Res. and Hum. Retrovir. 9: 483-487 (1993)). These models include mice, monkeys, and cats. Even though these animals are not naturally susceptible to HIV disease, chimeric mice models (e.g., SCID, bg/nu/xid, bone marrow-ablated BALB/c) reconstituted with human peripheral blood mononuclear cells (PBMCs), lymph nodes, or fetal liver/thymus tissues can be infected with HIV, and employed as models for HIV pathogenesis and gene therapy. Similarly, the simian immune deficiency virus (SIV)/monkey model can be employed, as can the feline immune deficiency virus (FIV)/cat model. Mice expressing siRNAs against hepatitis C RNA have demonstrated that siRNAs can work in a living mammal to prevent viral replication (McCaffrey, et al., Nature 418:38-39 (2002)). Similarly, to induce a patient to manufacture dual functional oligonucleotides, the patient's cells (e.g., bone marrow cells), can be transfected with plasmids encoding dual-functional oligonucleotide and reintroduced into the patient's body.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. An RNA-silencing agent having the following formula:
T-L-Vμ,
wherein T is an mRNA targeting moiety, L is a linking moiety, and Vμ is a viral miRNA recruiting moiety, forming the RNA-silencing agent.

2. An RNA silencing agent suitable for use in RNA silencing of a target mRNA, comprising:
a. an mRNA targeting portion complementary to the target mRNA;
b. a viral miRNA recruiting portion complementary to a viral miRNA; and
c. a linking portion that links the mRNA targeting portion and the viral miRNA recruiting portion.

3. The agent of claim 1 or 2, wherein the viral miRNA recruiting moiety recruits a viral miRNA.

4. The agent of claim 3, wherein the viral miRNA recruiting moiety recruits a RISC complex.

5. The agent of claim 2, wherein the RNA silencing agent is capable of mediating translational repression of the target mRNA.

6. The agent of claim 2, wherein the RNA silencing agent is capable of mediating cleavage of the target mRNA.

7. The agent of claim 1 or 2, wherein the viral miRNA is expressed by a virus selected from the group consisting of a double-stranded DNA virus, a single-stranded DNA virus, a double-stranded RNA virus, a double-stranded RNA virus, a single-stranded (plus-strand) virus, a single-stranded (minus-strand) virus, and a retrovirus.

8. The agent of claim 1 or 2, wherein the viral miRNA is expressed by a virus capable of infecting a mammalian cell.

9. The agent of claim 8, wherein the virus is capable of infecting a human cell.

10. The agent of claim 1 or 2, wherein the viral miRNA is expressed by a virus belonging to a family selected from the group consisting of Herpesviridae, Poxyiridae, Adenoviridae, Papillomaviridae, Parvoviridae, Hepadnoviridae, Retroviridae, Reoviridae, Filoviridae, Paramyxoviridae, Pneumoviridae, Rhabdoviridae, Orthomyxoviridae, Bunyaviridae, Hantaviridae, Picornaviridae, Caliciviridae, Togaviridae, Flaviviridae, Arenaviridae, Coronaviridae, and Hepaciviridae.

11. The agent of claim 1 or 2, wherein the viral miRNA is expressed by Human Immunodeficiency Virus (HIV).

12. The agent of claim 1 or 2, wherein the viral miRNA is expressed by a herpesvirus or an adenovirus.

13. The agent of claim 1 or 2, wherein the viral miRNA is expressed by a virus selected from the group consisting of Kaposi's Sarcoma-Associated Virus, Epstein Barr Virus, and Human Cytomegalovirus.

14. The agent of claim 1 or 2, wherein the viral miRNA is selected from the miRNA listed in Table 1.

15. The agent of claim 1 or 2, wherein the viral miRNA is derived from miRNA precursor selected from the group consisting of a pri-miRNA, a pre-miRNA, or a svRNA.

16. The agent of claim 15, wherein the svRNA is selected from the group consisting of VA-RNAI, VA-RNAII, EBER 1, EBER 2, MHV-68, CMER, RRE, TAR, POLADS, PAN RNA and IRES.

17. The agent of claim 1 or 2, wherein the mRNA targeting moiety or portion targets a viral mRNA.

18. The agent of claim 17, wherein the viral mRNA encodes a protein selected from the group consisting of a viral capsid protein, a viral envelope protein, a viral enzyme affecting interaction of the virus with a host cell, a viral transcriptase, an enzyme adding specific terminal groups to viral mRNA, an enzyme involved in integrating viral DNA into the host chromosome, an enzyme involved in processing viral or host nucleic acids, an enzyme involved in the modification or processing of a viral protein, a viral proteins required for modifying a host response to a virus, and a viral protein which can cause host cell death or lysis.

19. The agent of claim 1 or 2, wherein the mRNA targeting moiety or portion targets an mRNA encoding a host cell protein involved in a viral life cycle.

20. The agent of claim 19, wherein the host cell protein is involved in viral replication.

21. The agent of claim 19, wherein the host cell protein is involved in viral endocytosis.

22. The agent of claim 1 or 2, wherein the mRNA targeting moiety or portion targets an HIV mRNA.

23. The agent of claim 22, wherein the HIV mRNA is selected from the group consisting of gag, env, pol, tat, rev, vpu, vpr, vif, and nef

24. The agent of claim 1 or 2, wherein the mRNA targeting moiety or portion targets a host cell protein involved in a viral life cycle.

25. The agent of claim 24, wherein the protein is selected from the group consisting of CXCR4, CCR5, CD4, CyPA, Sam68, hRIP, Furin, and Tsg101.

26. The agent of claim 1 or 2, wherein the linking moiety or portion comprises a phosphodiester bond.

27. The agent of claim 1 or 2, wherein the linking moiety or portion comprises at least one modified nucleotide which increases the in vivo stability of the agent.

28. The agent of claim 27, wherein the linking moiety or portion comprises at least one 2′-O-methyl nucleotide, at least one peptide nucleic acid, or at least one locked nucleic acid.

29. A DNA construct encoding the RNA-silencing agent of any one of the preceding claims.

30. A composition comprising the RNA-silencing agent of claim 1 or 2 and a pharmaceutically acceptable carrier.

31. A method of treating a viral infection, comprising contacting a cell infected with a virus with the RNA-silencing agent of claim 1 or 2, thereby treating the viral infection.

32. A method of preventing propogation of a virus, comprising contacting a cell infected with the virus with the RNA-silencing agent of claim 1 or 2, thereby preventing propagation of the virus.

33. A method of treating or preventing a disease or disorder associated with a virus, comprising administering to a subject having the disease or disorder or at risk of having the disease or disorder with the RNA-silencing agent of claim 1 or 2, treating or preventing the disease or disorder.

34. Use of the RNA silencing agent of claim 1 or 2 in the manufacture of a medicament for repressing mutant or normal gene expression.
